(19) United States
(12) Patent Application Publication
(10) Pub. No.: US 2025/0207170 A1
(43) Pub. Date: Jun. 26, 2025

(54) CARBON CAPTURE IN TWO-STAGE FERMENTATION

(71) Applicant: LanzaTech, Inc., Skokie, IL (US)

(72) Inventors: Jennifer Rosa Holmgren, Skokie, IL (US); Sean Dennis Simpson, Auckland (NZ); Michael Koepke, Chicago, IL (US); Michael Emerson Martin, Chicago, IL (US); Zarath Morgan Summers, Prospect Heights, IL (US)

(21) Appl. No.: 18/987,199

(22) Filed: Dec. 19, 2024

Related U.S. Application Data
(60) Provisional application No. 63/613,518, filed on Dec. 21, 2023.

(51) Int. Cl. C12P 39/00

(52) U.S. Cl. CPC C12P 39/00

(57) ABSTRACT

The disclosure relates to a method for producing at least one product from CO and optionally H₂, or from CO₂ and H₂. The method comprises introducing a gaseous substrate comprising at least CO and optionally H₂, or at least CO₂ and H₂ into a first-stage bioreactor containing a culture of at least one microorganism in a liquid nutrient medium and fermenting the gaseous substrate to produce one or more first-stage product; and incorporating the one or more first products into one or more articles or converting the one or more first products into one or more second products. The converting may be a second stage fermentation process. The fermentations may be any combination of aerobic and anaerobic, batch and continuous.

### CROSS REFERENCE TO RELATED APPLICATIONS

This application claims the benefit of U.S. Provisional Patent Application No. 63/613,518 filed on Dec. 21, 2023, the entirety of which is incorporated herein by reference.

### FIELD

The disclosure relates to the combination of a first-stage fermentation that converts gas to first product with a second-stage fermentation that converts the first-stage product to a second-stage product. The first-stage product may be acetate, ethanol, or a mixture thereof, and the second-stage product may be any of a number of fermentation products generated from an ethanol and/or acetate feedstock. A portion of the first-stage product and/or the second-stage product may be incorporated into one or more articles or may be used in a chemical reaction to generate yet a further product.

### BACKGROUND

Mitigation of impending climate change requires drastic reductions in emissions of greenhouse gases (GHGs), such as those generated through the burning of fossil fuels like coal and oil. Climate change has also created an urgent demand for the development of sustainable food from renewable resources. Sustainable sources of food, chemicals and transportation fuels are currently insufficient to significantly displace our dependence on fossil carbon, and gas fermentation has recently emerged as an alternative platform for the biological fixation of gases such as carbon dioxide (CO₂), carbon monoxide (CO), and/or hydrogen (H₂) into sustainable fuels and chemicals. In particular, gas fermentation technology can utilize a wide range of feedstocks including gasified organic matter (for example, municipal solid waste or agricultural waste) or industrial waste gases (for example, from steel mills or oil refineries) to produce ethanol, acetate, and a variety of other products. Gas fermentation alone could displace 30% of crude oil use and reduce global CO₂ emissions by 10%, but as with any disruptive technology, many technical challenges remain before this potential is fully achieved.

The present disclosure provides system(s) and/or method(s) that provides the public with new methods for improved and/or increased production of a variety of useful products, including commodity chemicals from carbon capture and utilization.

### SUMMARY

It is against the above background that the present invention provides certain advantages and advancements over the prior art.

Although this invention disclosed herein is not limited to specific advantages or functionalities, the invention provides a two-step fermentation method for producing a product comprising culturing a first microorganism under conditions wherein the first microorganism ferments a first feedstock to produce an intermediate and culturing a second microorganism under conditions wherein the second microorganism ferments a second feedstock to produce the product from the intermediate.

In one embodiment the disclosure is a method for producing at least one product from CO and optionally H₂, or from CO₂ and H₂, the method comprising introducing a gaseous substrate comprising at least CO and optionally H₂, or at least CO₂ and H₂ into a first-stage bioreactor containing a culture of at least one first-stage microorganism in a liquid nutrient medium and fermenting the gaseous substrate to produce one or more first-stage products; and introducing at least a portion of at least one of the first-stage products to a second-stage bioreactor containing a culture of at least one second-stage microorganism in a liquid nutrient medium and fermenting the first-stage product to produce one or more second-stage products.

In some embodiment the disclosure is a method for the circular production of a commodity chemical, the method comprising passing a gaseous substrate to a first bioreactor containing a culture of a recombinant C1-fixing microorganism capable of producing at least one first product in a culture medium such that the microorganism converts the gaseous substrate to the at least one first product; and recovering the at least one first product from the bioreactor; passing a gaseous substrate to a second bioreactor containing a culture of a recombinant C1-fixing microorganism capable of producing at least one second product selected from ethylene and olefins, or any combination thereof in a culture medium such that the microorganism converts the gaseous substrate to the at least one second product; and recovering the at least one second product from the bioreactor; polymerizing the at least one first product with the at least one second product in the presence of a suitable polymerization catalyst to form a hybrid polymer; and converting the hybrid polymer into a tire.

In some embodiment, the disclosure is a method for producing at least one product from C1 carbon source including methane, the method comprises introducing a gaseous substrate comprising at least CO and optionally H₂, or at least CO₂ and H₂ into a first-stage bioreactor containing a culture of at least one first-stage microorganism in a liquid nutrient medium and fermenting the gaseous substrate to produce one or more first-stage products; and introducing at least a portion of at least one of the first-stage products to a second-stage bioreactor containing a culture of at least one second-stage microorganism in a liquid nutrient medium and fermenting the first-stage product to produce one or more second-stage products.

Other embodiments and options of operation are possible. Some of these are discussed in more detail below.

### BRIEF DESCRIPTION OF THE DRAWINGS

FIG. 1 is a diagram of an embodiment of the disclosure having a two-stage fermentation system for production of acetate, ethanol, or a mixture thereof in a first stage fermentation from CO and optionally H₂, or CO₂ and H₂, followed by a second-stage fermentation to produce an alcohol and CO₂. The CO₂ stream is recycled from the second-stage fermentation to the first-stage fermentation. Optionally, the CO₂ stream is treated to remove oxygen and form a substantially oxygen-free CO₂ stream.

FIG. 2 is a diagram of an embodiment of the disclosure having a two-stage fermentation system for production of acetate, ethanol, or a mixture thereof in a first stage fermentation from CO and optionally H₂, or CO₂ and H₂, followed by a second-stage fermentation to produce an alcohol and CO₂. A water electrolyzer is used to generate at least a portion of the H₂ used in the first-stage fermentation and optionally at least a portion of the O₂ generated by the water electrolyzer used in the second-stage fermentation.

FIG. 3A is a schematic showing a first microorganism (organism 1) that is a C1-fixing microorganism capable of producing an intermediate from a gaseous substrate and (2) a second microorganism (organism 2) that is a carbohydrate-fermenting microorganism capable of producing a product from the intermediate produced by the first microorganism (organism 1). FIG. 3B is a schematic showing a first microorganism (organism 1) that is a carbohydrate-fermenting microorganism capable of producing an intermediate from a sugar and a second microorganism (organism 2) that is a C1-fixing microorganism capable of producing a product from the intermediate produced by the first microorganism (organism 1).

FIG. 4 is a schematic showing a two-bioreactor fermentation process.

FIG. 5A is a schematic showing (1) a C1-fixing step for the production of acetone (an intermediate) from a C1-substrate and (2) a carbon-oxidizing process that converts acetone into a final product, isobutylene. FIG. 5B is a schematic showing (1) a C1-fixing step for the production of β-hydroxyisovaleric acid (an intermediate) from a C1-substrate and (2) a carbon-oxidizing step that converts β-hydroxyisovaleric acid into a final product, isobutylene.

FIG. 6 is a schematic showing (1) a C1-fixing step for the production of mevalonate (an intermediate) from a C1-substrate and (2) a carbon-oxidizing step that converts mevalonate into a final terpene product, such as isoprene or an isoprenoid.

FIG. 7A is a schematic showing a first microorganism that is a carbohydrate-fermenting microorganism capable of producing carboxylic and/or dicarboxylic acids from a sugar and a second microorganism that is a C1-fixing microorganism capable of producing alcohols and/or diols from the carboxylic and/or dicarboxylic acids produced by the first microorganism.

FIG. 7B is a schematic showing a first microorganism that is a carbohydrate-fermenting microorganism capable of producing saturated and/or unsaturated fatty acids from a sugar and a second microorganism that is a C1-fixing microorganism capable of producing saturated and/or unsaturated fatty alcohols from the saturated and/or unsaturated fatty acids produced by the first microorganism.

### DESCRIPTION

The disclosure relates to the combination of a primary fermentation (first stage fermentation) that converts gas to one or more first stage product, with a secondary fermentation (second stage fermentation) that converts one or more first product to one or more second stage product. In particular, the disclosure relates to a method for producing at least one product from CO and optionally H₂, or from CO₂ and H₂ or C1 carbon source including methane. The method comprises introducing a gaseous substrate comprising at least CO and optionally H₂, or at least CO₂ and H₂ into a first-stage bioreactor containing a culture of at least one first-stage microorganism in a liquid nutrient medium and fermenting the gaseous substrate to produce one or more first-stage products; and introducing at least a portion of at least one of the first-stage products to a second-stage bioreactor containing a culture of at least one second-stage microorganism in a liquid nutrient medium and fermenting the first-stage product to produce one or more second-stage products.

In an embodiment, the at least one of the one or more first-stage products and or at least one of the one or more second-stage products are incorporated into one or more articles or chemically converted into one or more final products. In an embodiment, one or more articles is an end user product.

### Primary Fermentation

The primary fermentation uses a microorganism (the “primary microorganism”) capable of converting gas to one or more first products. The one or more first product is selected from ethanol, isopropanol, monoethylene glycol, sulfuric acid, propylene, sodium hydroxide, sodium carbonate, ammonia, benzene, acetic acid, acetate, ethylene, ethylene oxide, formaldehyde, methanol or any combination thereof. In one embodiment, the first product is ethanol, acetate or mixture thereof. The primary fermentation may be a co-culture of two such organisms, which have similar or complementary growth requirements. Thus, the primary fermentation may involve a pure culture or a mixed culture.

The primary microorganism may, for example, be selected from Acetobacterium, Acetoanaerobium, Alkalibaculum, Blautia, Butyribacterium, Clostridium, Desulfobacterium, Eubacterium, Methanosarcina, Moorella, Oxobacter, Peptostreptococcus, Pyrococcus Ruminococcus, Sporomusa, and Thermoanaerobacter. In particular, the primary microorganism may be derived from a parental bacterium selected from Acetobacterium woodii, Alkalibaculum bacchii, Blautia producta, Butyribacterium methylotrophicum, Carboxydothermus acetogenium, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui. The primary microorganism may also be selected from Acetitomaculum ruminis, Acetoanaerobium noterae, Acetobacterium bakii, Acetobacterium carbinolicum, Acetobacterium dehalogenans, Acetobacterium fimetarium, Acetobacterium malicum, Acetobacterium paludosum, Acetobacterium tundrae, Acetobacterium wieringae, Acetobacterium woodii, Acetohalobium arabaticum, Acetonema longum, Blautia coccoides, Blautia hydrogenotrophica, Blautia producta, Blautia schinkii, Butyribacterium methylotrophicum, Clostridium aceticum, Clostridium autoethanogenum, Clostridium carboxidivorans, Clostridium drakei, Clostridium formicoaceticum, Clostridium glycolicum, Clostridium Clostridium mayombei, Clostridium methoxybenzovorans, Clostridium ragsdalei, Clostridium scatologenes, Eubacterium aggregans, Eubacterium limosum, Moorella mulderi, Moorella thermoacetica, Moorella thermoautotrophica, Oxobacter pfennigii, Sporomusa acidovorans, Sporomusa aerivorans, Sporomusa malonica, Sporomusa ovata, Sporomusa paucivorans, Sporomusa rhizae, Sporomusa silvacetica, Sporomusa sphaeroides, Sporomusa termitida, Thermoacetogenium phaeum, Thermoanaerobacter kivui, Acetobacterium, Moorella, Moorella sp HUC22-1, Moorella thermoacetica, Clostridium, Clostridium carboxidivorans, Clostridium drakei, Clostridium acidi-urici, Pyrococcus, Pyrococcus furiosus, Eubacterium, Eubacterium limosum, Desulfobacterium, Carboxydothermus, Acetogenium, Acetoanaerobium, Butyribacterium, Butyribacterium methylotrophicum, Peptostreptococcus, Ruminococcus, Oxobacter, Oxobacter pfennigii, Methanosarcina, Carboxydothermus, Eubacterium limosum, Desulfotomaculum orientis, Peptococcus glycinophilus, Peptococcus magnus, Ignicoccus hospitalis, Thermoanaerobacter kivui, and Thermoacetogenium phaeum. The microorganism may also be selected from those listed in Table 1 of Schiel-Bengelsdorf, FEBS Letters 586:2191-2198, 2012.

In one embodiment, the primary microorganism is Acetobacterium woodii, Clostridium autoethanogenum, Clostridium ljungdahlii, or Clostridium ragsdalei.

In one embodiment, the primary microorganism is a Wood-Ljungdahl microorganism. “Wood-Ljungdahl” refers to the Wood-Ljungdahl pathway of carbon fixation as described, for example, by Ragsdale, Biochim Biophys Acta, 1784:1873-1898, 2008. “Wood-Ljungdahl microorganisms” refers to microorganisms containing the Wood-Ljungdahl pathway, that expresses the enzymes of the pathway. The primary microorganism often contains a native Wood-Ljungdahl pathway.

In one embodiment, the primary microorganism is an acetogen. “Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784:1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3rd edition, p. 354, New York, NY, 2006). All naturally occurring acetogens are C1-fixing, anaerobic, autotrophic, and non-methanotrophic.

The primary microorganism is capable of consuming a substrate (a “primary substrate”) that provides carbon and/or energy. Typically, the primary substrate is gaseous and comprises a C1-carbon source, for example, CO, CO₂, and/or CH₄. In one embodiment, the primary substrate comprises a C1-carbon source of CO or CO+CO₂. The primary substrate may further comprise other non-carbon components, such as H₂, N₂, or electrons.

In one embodiment, the primary substrate comprises CO₂ and H₂. In another embodiment, the H₂ is renewable H₂. For example, the primary substrate may comprise about 1-80 or 1-30 mol % CO₂. In some embodiments, the primary substrate may comprise less than about 20, 15, 10, or 5 mol % CO₂. The primary substrate may comprise about 1, 2, 5, 10, 15, 20, or 30 mol % H₂. In some embodiments, the primary substrate may comprise a relatively high amount of H₂, such as about 60, 70, 80, or 90 mol % H₂. The primary substrate may also comprise some amount of CO and/or some amount of inert gases, such as N₂.

The primary substrate may be a waste gas obtained as a byproduct of an industrial process or from some other source, such as from automobile exhaust fumes or biomass gasification. In certain embodiments, the industrial process is selected from ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the substrate and/or C1-carbon source may be captured from the industrial process before it is emitted into the atmosphere, using any convenient method.

The primary substrate may be also syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste.

The composition of the substrate may have a significant impact on the efficiency and/or cost of the reaction. For example, the presence of oxygen (O₂) may reduce the efficiency of an anaerobic fermentation process, and oftentimes the primary fermentation will be anaerobic. Depending on the composition of the substrate, it may be desirable to treat, scrub, or filter the substrate to remove any undesired impurities, such as toxins, undesired components, or dust particles, and/or increase the concentration of desirable components.

In certain embodiments a CO₂ electrolyzer may be used to generate at least a portion of the CO used as primary substrate and optionally a portion of any O₂ used in the secondary fermentation.

In certain embodiments, the primary fermentation is performed in the absence of carbohydrate substrates, such as sugar, starch, lignin, cellulose, or hemicellulose.

The primary fermentation produces at least one product (a “first stage product”). Typically, this product is acetate, although the primary fermentation may also produce additional products such as ethanol, lactate, and 2,3-butanediol. In an embodiment the first stage product comprises ethanol, acetate or mixture thereof. Microbial biomass, such as single cell protein, may also be considered a product, as it has potential applications in animal feed and fertilizers. Importantly, the terms “acetate” and “acetic acid” may be used interchangeably herein.

Acetate plays a key role in global carbon cycle. 100 billion tons of acetate is metabolized annually in terrestrial habitats such as soils and sediments. 10 billion tons of acetate is produced each year by acetogens (such as Clostridium autoethanogenum). C. autoethanogenum can also produce ethanol/acetate mixtures from CO₂/H₂. High acetate production rates from CO₂+H₂ have been achieved, but acetate is toxic at high levels and low pH is required for efficient separation. Nearly all organisms are capable of utilizing acetate, including industrial workhorses E. coli, Clostridium, Corynebacterium, microalgae even though optimized for sugar conversion. Many organisms (including E. coli and yeast) equipped with both sets of genes for acetate uptake (pta-ack) and ethanol uptake (ald-adh/adhE). Many organisms have microcompartments for ethanol utilization. There should be no reason for parallel consumption not possible but may require some deregulation.

In one embodiment of the invention, the stream exiting the first bioreactor is treated to provide a purified or concentrated acid stream to be passed to the second bioreactor. In one embodiment of the invention the product of anaerobic fermentation are concentrated from the fermentation broth before being transferred to a second bioreactor containing a culture of at least one second microorganism in a second liquid nutrient medium, wherein the second microorganism is a different species from the first microorganism, and fermenting the first product to produce at least a first target product in a second fermentation broth. In another embodiment, a concentration step or purification step is utilized on the primary fermentation product before the primary fermentation product is transferred to a second fermentation. In one embodiment, the product of anaerobic fermentation is subject to a treatment step before being transferred to a second fermentation culture. In some embodiments, the treatment step comprises a concentration step, purification step, isolation and purification steps, or any combination thereof. In one embodiment, the treatment step may be performed by using any method or combination of methods known in the art, including, for example, fractional distillation, chromatography, evaporation, pervaporation, gas stripping, phase separation, and extractive fermentation, including for example, liquid-liquid extraction. In one embodiment, the first product is a mixture comprising ethanol and acetate. The step of purification includes, not limited to, affinity purification, filtration, centrifugation, evaporation, liquid-liquid extraction, crystallisation, recrystallisation, trituration, adsorption, chromatography, refining, distillation, downstream processing, fractionation, electrolysis, sublimation, bioleaching, plasma chemical purification. The step of isolation includes, not limited to, centrifugation, cyclonic separation, chelation, chromatography including high-performance liquid chromatography, thin layer chromatography, counter current chromatography, droplet counter current chromatography, paper chromatography, ion chromatography, size exclusion chromatography, affinity chromatography, centrifugal partition chromatography, gas chromatography and inverse gas chromatography; crystallisation, decantation, demister, distillation, drying, electrophoresis, electrostatic separation, elutriation, evaporation, extraction including leaching, liquid-liquid extraction, solid phase extraction, supercritical fluid extraction, subcritical fluid extraction, field flow fractionation, mesh filtration, flocculation, fractional distillation, fractional freezing, magnetic separation, oil-water separation, precipitation, recrystallisation, scrubbing, sedimentation, sieving, stripping, sublimation, vapour-liquid separation, winnowing, zone refining etc. In one embodiment, when the first product is a mixture of ethanol and acetate, the treatment step comprises a concentration step carried out by chromatography, ion exchange chromatography, or chromatography resins.

In some embodiments the primary fermentation may produce one or more of ethanol (WO 2007/117157), acetate (WO 2007/117157), 1-butanol (WO 2008/115080, WO 2012/053905, and WO 2017/066498), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2017/066498), 1-hexanol (WO 2017/066498), 1-octanol (WO 2017/066498), chorismate-derived products (WO 2016/191625), 3-hydroxybutyrate (WO 2017/066498), 1,3-butanediol (WO 2017/066498), 2-hydroxyisobutyrate or 2-hydroxyisobutyric acid (WO 2017/066498), isobutylene (WO 2017/066498), adipic acid (WO 2017/066498), 1,3-hexanediol (WO 2017/066498), 3-methyl-2-butanol (WO 2017/066498), 2-buten-1-ol (WO 2017/066498), isovalerate (WO 2017/066498), isoamyl alcohol (WO 2017/066498), and monoethylene glycol (WO 2019/126400).

### Secondary Fermentation

The secondary fermentation uses a microorganism (the “secondary microorganism”) capable of converting ethanol, acetate or mixture thereof to one or more second stage product. The secondary fermentation may be a co-culture of two such organisms, which have similar or complementary growth requirements. Thus, the primary fermentation may involve a pure culture or a mixed culture. The secondary fermentation may be conducted as an anaerobic or aerobic process.

The secondary microorganism may be any microorganism capable of consuming ethanol, acetate or mixture thereof. The secondary microorganism may be a yeast or bacterial species. The secondary microorganism may be selected from those listed in the following table:

| | | Typical | | |
|---|---|---|---|---|
| | Used | fermentation | | |
| Exemplar species | industrially | type | Oxygenation | Known products |
| Escherichia coli | Yes | Batch | Any | Proteins, base chemicals, |
| | | | | fine chemicals |
| Yarrowia lipolytica | Yes | Continuous | Aerobic | Bulk and fine |
| | | | | [supplement] lipids |
| Saccharomyces | Yes | Batch | Anaerobic | Ethanol, dried distiller's |
| cerevisiae | | | | grains and solubles |
| Saccharomyces | Yes | Batch | Aerobic | Biomass, proteins, Base |
| cerevisiae | | | | Chemicals, fine |
| | | | | chemicals |
| Geobacillus | Yes | Batch | Aerobic | Enzymes [amylase] |
| thermoglucosidasius | | | | |
| Bacillus subtilis | Yes | | Aerobic | Enzymes [decarboxylase, |
| | | | | glucanase, hemicellulase, |
| | | | | amylase, xylanse], |
| | | | | bacteriocins, amino acids |
| Pseudomonas putida | No | Batch | Aerobic | Proteins, base chemicals, |
| KT2440 | | | | fine chemicals |
| Cryptococcus curvatus | No | Continuous | Aerobic | Lipids |
| Rhodotorula glutinis | No | Continuous | Aerobic | Lipids |
| Lipomyces starkeyi | No | Continuous | Aerobic | Lipids |
| Cupriavidus | No | Batch | Aerobic | Biomass, PHBs, PHAs, |
| necator/Ralstonia | | | | isobutanol |
| eutropha/Alcaligenes | | | | |
| eutrophus | | | | |
| Clostridium kluyveri | Yes | Continuous | Anaerobic | Butanoate, hexanoate, |
| | | | | octanoate, propionate |
| Methanosarcina | Yes | Continuous | Anaerobic | Methane, |
| acetivorans | | | | biomass/digestate |
| Desulfonatronum | No | Batch | Aerobic | N/A |
| cooperativum | | | | |
| Geoalkalibacter | No | Batch | Aerobic | N/A |
| ferrihydriticus | | | | |
| Alkalispirillum mobile | No | Batch | Aerobic | N/A |
| Corynebacterium | Yes | Batch | Aerobic | Amino acids, biomass, |
| glutamicum | | | | alcohols on research |
| | | | | scale |

The secondary microorganism is capable of consuming a substrate (a “secondary substrate”) that provides carbon and/or energy. Typically, the secondary substrate comprises the ethanol, acetate or mixture thereof produced by the primary fermentation. However, the secondary substrate may also contain additional sources of carbon and/or energy, such as sugar or starch. In specific embodiments, acetate provides at least 50%, at least 60%, at least 70%, at least 80%, at least 90%, at least 95%, or at least 99% of the carbon and/or energy requirements of the secondary microorganism. In some embodiments, however, acetate may be removed for another use and another substrate may drive the secondary fermentation. The second fermentation produces at least one product, which may be referred to herein as a “second stage product.” The second stage product may be an enzyme, such as alpha acetolactate decarboxylase, alpha amylase, catalase, chymosin, cyclodextrin glucanotransferase, beta glucanase, glucose isomerase, glucose oxidase, hemicellulase, lipase, maltogenic amylase, protease, pullulanase, xylanase, or cellulase. The second stage product may be a nutrient or additive, such as microbial biomass for example, single cell protein), amino acids (glutamate, lysine, threonine, phenylalanine, tryptophan, cysteine, valine, proline, arginine, histidine), lipids, or vitamins (B12, B2, biotin). The second stage product may be a chemical such as butanoate, hexanoate, octanoate, isobutanol, isoprene, terpenes or terpenoids. The second stage product may also be, for example, methane, polyhydroxybutyrate, polyhydroxyalkanoate, 3-hydroxyproprionate, a C₅-C₂₀ alcohol, an organic acid, a ketone, a lipid, acetone, propanol, butanol, 2,3-butanediol, acetate, butyrate, caproate, propylene, butadiene, isobutylene, isobutyl acetate, succinate, mevalonate, itaconic acid, monellin, or ethylene. In some cases the second stage product may be just one or more of these products devoid of others, for example, devoid of lipids. In one embodiment, the second stage product may be polyhydroxybutyrate devoid of lipids. In another embodiment it may be polyhydroxyalkanoate devoid of lipids.

The second stage product may also be, for example, any of the products described and depicted by Lee, Nature Catalysis, 2:18-33, 2019. The second product may be one or more of ethanol (WO 2007/117157, U.S. Pat. No. 7,972,824), acetate (WO 2007/117157, U.S. Pat. No. 7,972,824), 1-butanol (WO 2008/115080, U.S. Pat. No. 8,293,509, WO 2012/053905, U.S. Pat. No. 9,359,611 and WO 2017/066498, U.S. Pat. No. 9,738,875), butyrate (WO 2008/115080, U.S. Pat. No. 8,293,509), 2,3-butanediol (WO 2009/151342, U.S. Pat. No. 8,658,408 and WO 2016/094334, U.S. Pat. No. 10,590,406), lactate (WO 2011/112103, U.S. Pat. No. 8,900,836), butene (WO 2012/024522, US2012/045807), butadiene (WO 2012/024522, US 2012/045807), methyl ethyl ketone (2-butanone) (WO 2012/024522, US 2012/045807 and WO 2013/185123, U.S. Pat. No. 9,890,384), ethanol which is then converted to ethylene (WO 2012/026833, US 2013/157,322), acetone (WO 2012/115527, U.S. Pat. No. 9,410,130), isopropanol (WO 2012/115527 U.S. Pat. No. 9,410,130), lipids (WO 2013/036147 U.S. Pat. No. 9,068,202), 3-hydroxypropionate (3-HP) (WO 2013/180581, U.S. Pat. No. 9,994,878), terpenes, including isoprene (WO 2013/180584, U.S. Pat. No. 10,913,958), fatty acids (WO 2013/191567 U.S. Pat. No. 9,347,076), 2-butanol (WO 2013/185123 U.S. Pat. No. 9,890,384), 1,2-propanediol (WO 2014/036152, U.S. Pat. No. 9,284,564), 1-propanol (WO 2014/0369152, U.S. Pat. No. 9,284,564), 1 hexanol (WO 2017/066498, U.S. Pat. No. 9,738,875), 1 octanol (WO 2017/066498, U.S. Pat. No. 9,738,875), chorismate-derived products (WO 2016/191625, U.S. Pat. No. 10,174,303), 3-hydroxybutyrate (WO 2017/066498, U.S. Pat. No. 9,738,875), 1,3-butanediol (WO 2017/066498, U.S. Pat. No. 9,738,875), 2-hydroxyisobutyrate or 2-hydroxyisobutyric acid (WO 2017/066498, U.S. Pat. No. 9,738,875), isobutylene (WO 2017/066498, U.S. Pat. No. 9,738,875), adipic acid (WO 2017/066498, U.S. Pat. No. 9,738,875), 1,3-hexanediol (WO 2017/066498, U.S. Pat. No. 9,738,875), 3-methyl-2-butanol (WO 2017/066498, U.S. Pat. No. 9,738,875), 2-buten-1-ol (WO 2017/066498, U.S. Pat. No. 9,738,875), isovalerate (WO 2017/066498, U.S. Pat. No. 9,738,875), isoamyl alcohol (WO 2017/066498, U.S. Pat. No. 9,738,875), and/or monoethylene glycol (WO 2019/126400, U.S. Pat. No. 11,555,209) in addition to 2-phenylethanol (WO 2021/188190, US 2021/0292732) and ethylene.

In one embodiment, one or more second-stage products include alcohols, acids (ex., fatty acids and omega 3 fatty acids), biofuels, alternative proteins, food products, food additives and ingredients, industrial enzymes, vitamins and nutritional supplements, antibiotics and pharmaceuticals, bioplastics, textiles and fibers, flavor and fragrance compounds, biochemicals and specialty chemicals, biogas and biomethane, biochemicals for agriculture, biopharmaceuticals, animal health products, biodegradable polymers, cellular agriculture products, amino acids, cellulosic biofuels, bio-based chemical intermediates, feed products, beverage products, medicinal products, silk proteins, cultured protein, chemicals or mixture thereof. In one embodiment, the first stage product is acetic acid, and the second stage product is an acid ex. fatty acids, omega 3 fatty acids etc. When an acid is produced as the second stage product, the overall requirement of buffering is reduced or eliminated. In one embodiment, the primary bioreactor contains fermentation broth comprising a culture of one or more acetogenic bacteria in a liquid nutrient medium. A gaseous substrate comprising cither CO and optionally H₂, or CO₂ and H₂, or mixtures thereof, is supplied to the primary bioreactor, wherein the gas is converted by the bacteria to acetate (first product). The primary bioreactor is maintained at a pH in the range of 2.5-5, or 3-4, or 6.5-7. The acetate product leaves the primary bioreactor in an aqueous broth stream, which is treated to remove the biomass using known methods. The resulting acetate-containing treated stream is fed to a secondary bioreactor. In the secondary bioreactor, acetate in the treated stream is converted to one or more second products and biomass by a microorganism. Because the secondary fermentation consumes acetate, the pH of the broth will increase. However, when the second product is an acid such as fatty acid (example Omega 3 fatty acid) the requirement to adjust the pH of the broth for maintaining the culture of secondary microorganism is reduced or not required at all. The overall cost associated with acid buffering is reduced. Production of an acid as one or more second product significantly reduces the cost of fermentation pH control relative to a system in which pH is controlled by direct addition of acid to the bioreactor media.

In one embodiment, one or more first stage products produced according to the method of the disclosure may be used in numerous product applications, including preparing vinyl acetate, Vinyl acetate Monomer (VAM) for adhesives, water based paints, textile/paper coatings and films; Cellulose acetate for textiles, films and cigarette filters; acetate fibers for textile industry to make fabrics for clothing, upholstery and linens, packaging industry; acetic anhydride for pharmaceuticals, dyes and perfumes; acetaminophen (paracetamol); methyl acetate as solvent in paints, coatings and adhesives. It is also employed as a flavoring agent and as an environmentally friendly alternative to traditional solvents; vinegar; acetate plastic as it is a thermoplastic material it can be used in eyeglass frames, packaging materials and various consumer goods; dyes and pigments for textiles, paints and inks; ethyl cellulose for coating material, binder, and controller release agent in pharmaceutical, cosmetic and food industry; Food additives ex acetate salts as flavour enhancers, preservatives and acidity regulator; pharmaceuticals as solvent in production of pharmaceuticals including antibiotics, vitamins and other drugs; acetylcholine; acetyl coenzyme A in cellular metabolism; polyvinyl acetate (PVA) emulsions for adhesives, paints and coatings; acetic acid derivatives in production of dyes, perfumes and various pharmaceuticals; acetate esters such as butyl acetate, ethyl acetate, and methyl acetate as solvents, coatings and flavouring agents; acetone as solvent, for chemical manufacturing and plastic production; acetophenone for fragrances and flavours; acetonitrile as solvent and chemical in organic synthesis; acetic acid aluminium salt for topical medications and as an astringent; acetylated Starch for food and pharmaceutical industry as stabilizer and thickening agent; ethyl acetate peroxide as initiator in the polymerization of some monomers; ethyl acetate sulfate as surfactant and emulsifying agent; propyl acetate as solvent in coatings, paints and cleaning applications etc.

In one embodiment, one or more first-stage products, one or more second-stage products, one or more articles, and or one or more final products are selected from ethanol, acetic acid, butyric acid, propionic acid, methane, itaconic acid, 3-Hydroxypropionic acid, acrylic acid, vinyl acetate, malonic acid, acrylamide, propiolactone, oligomers, and polyesters, lactic acid, succinic acid, formic acid, mevalonic acid, tyrosine, phloroglucinol, fatty acids, omega-3 fatty acids, lipids, β-Caryophyllene, polyhydroxyalkanoates, isobutyl acetate, hydrogen, monellin, diacylglycerides, a C₅-C₂₀ alcohol, an organic acid, a ketone, a lipid, propanol, butanol, 2,3-butanediol, butyrate, propionate, caproate, butadiene, isobutylene, polyhydroxyalkanoates, succinate, mevalonate, vinyl acetate, Vinyl Acetate Monomer (VAM), cellulose acetate, acetate fibers, acetic anhydride, acetaminophen, methyl acetate, vinegar, acetate plastic, dyes, pigments, ethyl cellulose, food additives, pharmaceuticals, acetylcholine, acetyl coenzyme A, polyvinyl acetate (PVA) emulsions, acetic acid derivatives, acetate esters such as butyl acetate, ethyl acetate, and methyl acetate, acetone, acetophenone, acetonitrile, acetamide, acetylene, diethyl ether (ethoxyethane), acetic ether (ethyl acetate dimer), acetic acid anhydride (ethanoic anhydride), acetyl salicylic acid (Aspirin), acetyl cellulose, acetic acid esters of glycerol (Acetins), ethylene vinyl acetate (EVA), acetate ions, acetyl bromide, sodium acetate, acetate buffer, acetylferrocene, benzyl acetate, acetate dyes, acetic acid aluminium salt, acetylated starch, vinyl acetate copolymer, ethyl acetate peroxide, ethyl acetate sulfate, acetyl glucosamine, propyl acetate, acetyl glycine, acetic acid ammonium salt, acetic acid ethylene ester (Ethylidene Diacetate), acetic acid citrate solution, acetylcholinesterase, acetylcholinesterase inhibitors, acetylacetonate, acetylthiocholine, acetylated wood, acetyl fluoride, acetic acid lithium salt, ethyl acetate nitrate, acetyl triethyl citrate acetylated lanolin, acetaldehyde, ethyl acetoacetate, ethyl lactate, acetated alcohol or any combination thereof.

In one embodiment, the one or more first-stage products, one or more second-stage products, one or more articles, and or one or more final products is incorporated into or utilized as adhesives, water based paints, textile/paper coatings and films, textiles, cigarette filters, fabrics for clothing, upholstery and linens, packaging, pharmaceuticals, dyes and perfumes, solvent in paints, flavoring agent, thermoplastic material, eyeglass frames, various consumer goods, inks, coating material, binder, cosmetic, food, flavour enhancers, preservatives acidity regulator, solvent in production of pharmaceuticals, solvent, chemical manufacturing, plastic production, topical medications, astringent, food and pharmaceutical industry as stabilizer and thickening agent, initiator in the polymerization of some monomers, surfactant and emulsifying agent, solvent in coatings, paints and cleaning applications, cleaning products such as detergents, disinfectants, surface cleaners and sanitizers, biofuel, hand sanitizers, medicines, including liquid formulations, tinctures and syrups; extraction and dissolution of active ingredients, precursor or intermediate in the synthesis of various chemicals ex acetaldehyde, ethylene, ethyl acetate, acetic acid, and ethylene glycol, plastics, resins, fibers, surfactant and in the production of detergents, textiles and personal care products, resins, plastics, dyes and other chemicals, solvent in coatings, adhesives and automotive industries, extraction agent and in the production of pharmaceuticals and industrial chemicals, intermediate in synthesis of pharmaceuticals, cosmetics, dyes and flavors, biodegradable solvent used in paints coatings and personal care products, for the production of propiolactone, oligomers, and polyesters, manufacture clothing, linens, upholstery, and other textile products, food and beverages, cleaning products both for household and industrial cleaning agents, printing and photography, various industrial chemicals, fertilizer, superphosphates, detergents synthetic resins, dyestuffs, petroleum catalysts, insecticides, antifreeze, pigments, enamels, printing inks, coated fabrics, paper, cellophane, acetate textile, viscose textiles, lubricants, non-ferrous metals, batteries, anaesthetics, fruit ripening agents, fertilizers, safety glass, metal cutting gases, welding gases, high velocity thermal sprays, refrigerants, or any combination thereof.

In one embodiment, one or more first stage products and/or second stage products is a commodity chemical. In one embodiment, wherein the commodity chemical is selected from ethanol, acetate, isopropanol, monoethylene glycol, sulfuric acid, propylene, sodium hydroxide, sodium carbonate, ammonia, benzene, acetic acid, ethylene oxide, formaldehyde, methanol, or any combination thereof. In one embodiment, the commodity chemical is aluminum sulfate, ammonia, ammonium nitrate, ammonium sulfate, carbon black, chlorine, diammonium phosphate, monoammonium phosphate, hydrochloric acid, hydrogen fluoride, hydrogen peroxide, nitric acid, oxygen, phosphoric acid, sodium silicate, titanium dioxide, or any combination thereof. In another embodiment, the commodity chemical is acetic acid, acetone, acrylic acid, acrylonitrile, adipic acid, benzene, butadiene, butanol, caprolactam, cumene, cyclohexane, dioctyl phthalate, ethylene glycol, methanol, octanol, phenol, phthalic anhydride, polypropylene, polystyrene, polyvinyl chloride, polypropylene glycol, propylene oxide, styrene, terephthalic acid, toluene, toluene diisocyanate, urea, vinyl chloride, xylenes, or any combination thereof. The method according to one embodiment, wherein the commodity chemical is utilized in the sector selected from plastics, synthetic fibers, synthetic rubber, dyes, pigments, paints, coatings, fertilizers, agricultural chemicals, pesticides, cosmetics, soaps, cleaning agent, detergents, pharmaceuticals, mining, or any combination thereof.

In another embodiment, the method includes incorporating a commodity chemical into one or more articles or converting a commodity chemical into a product selected from ethanol, acetate, 1-butanol, butyrate, 2,3-butanediol, lactate, butene, butadiene, methyl ethyl ketone (2-butanone), ethylene, acetone, isopropanol, lipids, 3-hydroxyproprionate, terpenes, isoprene, fatty acids, 2-butanol, 1,2-propanediol, 1 propanol, 1 hexanol, 1 octanol, chorismate-derived products, 3-hydroxybutyrate, 1,3-butanediol, 1,4-butanediol, 2-hydroxyisobutyrate, 2-hydroxyisobutyric acid, isobutylene, adipic acid, 1,3-hexanediol, 3-methyl-2-butanol, 2-buten-1-ol, isovalerate, isoamyl alcohol, monoethylene glycol, antiseptic hand rubs, therapeutic treatments for methylene glycol poisoning, therapeutic treatments for methanol poisoning, pharmaceutical solvent for pain medication, oral hygiene products, antimicrobial preservative, engine fuel, rocket fuel, plastics, fuel cells, home fireplace fuels, industrial chemical precursor, cannabis solvent, winterization extraction solvent, paint masking product, paint, tincture, purification and extraction of DNA and RNA, cooling bath for various chemical reactions, ethylene to raw material, anaesthetic, ethylene and nitrogen in fruit ripening, fertilizer, safety glass, oxy-fuel in metal cutting, welding, high velocity thermal spraying, refrigerant, raw material to polyethylene, raw material to PET, raw material to PVC, fibers, packaging, coatings, adhesives, ethylene dichloride (EDC), vinyl chloride monomer (VCM), alpha olefins, linear alpha olefins, detergent alcohols, plasticizer alcohols, vinyl acetate monomer (VAM), barrier resins, industrial ethanol, ethyl acetate, ethyl acrylate, polyethylene oligomers, Ultra-high-molecular-weight polyethylene (UHMWPE), Ultra-low-molecular-weight polyethylene (ULMWPE or PE-WAX), High-molecular-weight polyethylene (HMWPE), High-density polyethylene (HDPE), High-density cross-linked polyethylene (HDXLPE), Cross-linked polyethylene (PEX or XLPE), Medium-density polyethylene (MDPE), Low-density polyethylene (LDPE), Very-low-density polyethylene (VLDPE), Chlorinated polyethylene (CPE), films, food packaging, non-food packaging, shrink film, stretch film, containers, drums, household goods, caps, pallets, pipes, refuse sacks, carrier bags, industrial lining, ethylene oxide, ethoxylates, shampoo, kitchen cleaners, glycol ethers, ethanolamines, surfactants, personal care products, pet care products, candles, car care products, polyester fibers, textiles, nonwovens, cover stock for diapers, road-building fabrics, filters, fiberfill, felts, transportation upholstery, paper reinforcement, tape reinforcement, tents, rope, cordage, sails, fish netting, seatbelts, laundry bags, synthetic artery replacements, carpets, rugs, apparel, sheets, pillowcases, towels, curtains, draperies, bed ticking, blankets, liquid coolant, antifreeze, preservative, dehydrating agent, drilling fluid, polyester resins, insulation materials, polyester film, de-icing fluid, heat transfer fluid, automotive antifreeze, water-based adhesives, latex paints, asphalt emulsions, electrolytic capacitors, synthetic leather, polyester resin PET, jars, bottles, plastic bottles, high-strength fibers, Dacron, durable-press blends, insulated clothing, furniture filling, pillow filling, artificial silk, carpet fiber, automobile tire yarns, conveyor belts, drive belts, reinforcement for fire and garden hoses, nonwoven fabrics for stabilizing drainage ditches, culverts, railroad beds, nonwovens for diaper topsheets, disposable medical garments, high-strength plastics, magnetic recording tape, photographic film, as feedstock, solvents for cosmetics, inks, medicinal tablets, disinfectants, sterilizers, skin creams, purification of vegetable oil and fats, purification of animal oil and fats, cleaning agent, drying agent, aerosol solvent, derivative ketones, isopropylamines, isopropyl esters, propylene, polypropylene oligomers, polymerization modifier, coupling agent, heat resistant articles, kettles, food containers, disposable bottles, clear bags, flooring, mats, adhesive stickers, foam polypropylene, building materials, hydrophilic clothing, medical dressings, or any combination thereof.

In another embodiment, the method includes incorporating a commodity chemical into an article or converting a commodity chemical into a product selected from humectants, filters, fire extinguishing sprinkler system, fuel for warming foods, heat transfer fluids, non-reacted component in formulation, deodorizing or air purifying, softening agent, arts/craft glue/paste, toys, children products, freezer gel pack, treating wood rot and fungus, preserving biological tissues and organs, alkyd type resins, resin esters, enamels, lacquers, latex paint, asphalt emulsion, thermoplastic resin, hydrate inhibition agent, agent for removing water vapor, shoe polish, vaccines, screen cleaning solution, water-based hydraulic fluid, heat transfer for liquid cooled computers, personal lubricant, lubricant, toothpaste, anti-foaming agent in food industrial applications, flame resistant hydraulic fluids, additive for electrolytic polishing belts, industrial solvent, trash bags, shower curtains, cups, utensils, medical devices, durable goods, nondurable goods, plastic sacks, plastic lids, industrial strapping, construction materials, felt, ovenable trays, frozen food trays, microwavable tray, artificial vascular scaffolds, vascular prostheses, woven devices, polyester-based prostheses, vehicle liner material, soaps, cosmetic products, laundry detergent jugs, laundry detergent, soap microplastic, microbeads, cosmetic product microbeads, detergent pods, disinfectant with scrubbing agents, toothpaste with microbeads, face wash, conditioner, body wash, hand cleaner, exfoliating products, bath products, shower gels, powder laundry detergent, lotions, deodorants, toilet cleaners, sunscreen, shopping bags, mouthwash bottles, peanut butter containers, salad dressing and vegetable oil containers, polar fleece fiber, tote bags, paneling, milk jugs, juice bottles, bleach bottles, motor oil bottles, cereal box liners, recycling containers, floor tile, drainage pipe, benches, picnic tables, fencing, wire jacketing, sliding windows, decks, mud flaps, roadway gutters, speed bumps, squeezable bottles, bread, dry cleaning bags, trash can liners, trash cans, compost bins, shipping envelopes, lumber, syrup bottles, ketchup bottles, straws, medicine bottles, battery cables, battery cases, disposable plates and cups, egg cartons, carry-out containers, compact disc cases, signs and displays, synthetic fibers, yarn, stable phase change material, thermal energy storage material, nylon 6,6, nylon, tires, rubber, adiponitrile, shoes, footwear, or any combination thereof.

In one embodiment, the one or more first stage products, one or more second stage products, or any combination thereof is incorporated into or utilized as a personal care product, a fragrance, a cosmetic, or a home care product. Examples include perfume, cologne, aftershave, shampoo, shower gel, soap, body lotion, laundry detergents, fabric softeners, dryer sheets, room fresheners, carpet fresheners, essential oils, cleaning, polishing, scouring abrasives, toiletries, sanitary preparations, tissue, nail products, diapers, bandages, sunscreen, nourishing cream, hand cream, lipstick, lip gloss, sun oil, massage cream, cleansing cream, facial pack, serums for cosmetic purposes, cocoa butter, skin care product, retinol cream, muscle gel, body paint, cooling spray, cold cream, pomade, cleaning products, dishwashing liquid or any combination thereof.

The primary and secondary fermentations process to obtain acetate/ethanol or mixture thereof as first stage product and subsequent fermentation of the same in secondary fermentation is advantageous as compared to fermentation of sugar, as the same is not limited by the availability or supply of sugar.

### Integrated Design

The primary and secondary fermentations may be integrated in some way, with each other and/or with upstream or downstream processes and or technologies. In an embodiment, the primary fermentation is carried out in a first bioreactor and secondary fermentations is carried out in a second bioreactor. In some embodiments, both the primary and secondary fermentation are carried out in a single bioreactor. In some embodiments, a bioreactor comprises a co-culture of a first microorganism and a second microorganism. In some embodiments, while using single bioreactor, first microorganism is present in the reactor while carrying out primary fermentation. The second microorganism is present in the bioreactor while carrying out secondary fermentation.

One such integration integrates electrolysis with O₂ output. Both CO₂ electrolysis to produce CO, and water electrolysis to produce H₂, necessarily produce O₂ as a byproduct. In the context of two-stage fermentation, water electrolysis can be used to provide the H₂ in the H₂+CO₂ fermentation to make acetic acid occurring in the first stage fermentation. A large amount of O₂ may also be produced from the water electrolysis. The O₂ byproduct may not be pure and may not be an immediately saleable product. To sell the O₂ as industrial oxygen would require removal of trace gas components and moisture and would further require compression. However, the O₂ may be used in an aerobic fermentation, such as in the second stage fermentation, if it is an aerobic fermentation. Aerobic fermentation may be used to produce, for example: (1) cellulases by Trichoderma reesei or Aspergillus niger, as well as other species of filamentous fungi (growth on acetic acid on A. niger is documented, and co-consumption of glucose and acetic acid is documented on T. reesei) and (2) antibiotics (growth and production of penicillin is elevated when culture is provided acetic acid as well as glucose, pH 6.5).

If the second stage does not require oxygen or there is excess oxygen, the oxygen can be used for enhanced combustion in various refinery or steelmaking processes. The oxygen can also be used in gasification. Refinery burners with enhanced O₂ feed have greater efficiency (oxyfuel burners). Steel mills use waste gasses for heating cheaply for steel rolling or other processes, and as a basic oxygen furnace input.

Another integration involves the integration of nutrients between the primary and secondary fermentations. Excess vitamins, for example, B vitamins, could be transferred from stage 1 to stage 2. B vitamins help induce production of a broad range of hydrolytic enzymes in filamentous fungi. If the second stage fermentation uses yeast as a microorganism, yeast extract can provide carbon source and nutrients for growth. Yeast extract is also a nitrogen source (8 to 12 wt. % nitrogen) and can be a pH neutral source of N.

Another form of nutrient integration occurs when a particular nutrient that is required by both the first and second microorganisms for the first and second fermentations is added to the first nutrient medium in an amount that is sufficient for both fermentations. Thus, when the fermentation broth from the first bioreactor is transferred to the second bioreactor, one need not add that particular nutrient to the second bioreactor, or one need not add as much of the particular nutrient. Alternatively, the particular nutrient may not be required or utilized by the first microorganism and is merely present in the first fermentation without being utilized or consumed. In one embodiment, the first fermentation broth is transferred and utilized by the second fermentation's microorganism without the need to augment or alter its composition. Another aspect of nutrient integration involves recycling of the second fermentation broth back to the first fermenter so that remaining nutrients or products of the second fermentation can be utilized in the first fermentation. This aspect can be utilized in either the continuous culture or batch fed culture embodiments.

Another aspect of nutrient integration employs a nutrient in the first fermentation to induce or increase production of a product in the first fermentation that is useful for growth of the microorganism in the second fermentation and/or conversion by the microorganism in the second fermentation bioreactor. Thus, upon transfer of the first fermentation broth to the second bioreactor, the product in it stimulates biomass production in the second bioreactor or is fermented in the second bioreactor to form the first or a second target product.

In some embodiments of the disclosure, it is desirable that the first microorganism be removed from the first fermentation broth prior to transfer of the first fermentation broth to the second bioreactor. It may be desirable to recycle all or a portion of the first microorganism back to the first bioreactor. In other embodiments, the removed first microorganism may be used as biomass. Similarly, it may be desirable that the second microorganism be removed from the second fermentation broth prior to transfer of the second fermentation broth back to the first bioreactor. It may be desirable to use the removed second microorganism as biomass or to recycle the second microorganism back to the second bioreactor.

In one embodiment, the primary fermentation could be run at low pH, which could inhibit the growth of species from the secondary fermentation, if broth from the secondary fermentation were recycled back to the primary fermentation. This would also reduce acid compensation costs. The secondary fermentation could be run at a higher pH than the primary fermentation, ideally by more than 1 full pH unit. When acid is consumed in the first bioreactor, pH rises and so transfer of a higher pH second reactor fermentation broth to the first bioreactor reduces the amount of base, such as ammonia, that would need to be supplied to adjust the pH of the first bioreactor. Yeast, E. coli, and a wide range of microbes operate near pH 7 and can be used in the second bioreactor.

In another embodiment, an integration may be the recycling of tail gas from, for example, the secondary fermentation to the primary fermentation. The tail gas from one fermentation may contain CO₂ and/or H₂, which may be used as a substrate in the other fermentation. A challenge that may be faced with this integration occurs when the one of the bioreactors operates in an aerobic mode while the other operates in an anaerobic mode. For case of understanding, this embodiment is explained wherein the primary fermentation is anaerobic and the secondary fermentation is aerobic. O₂ present in the tail gas from the secondary bioreactor must be separated and removed from the CO₂ containing tail gas stream before the tail gas is recycled to the primary bioreactor. The amount of O₂ removed should be enough so that any remaining O₂ that is passed to the primary bioreactor does not adversely affect the operations of the primary bioreactor beyond tolerances. Suitable techniques for separation include pressure swing adsorption (PSA), membrane separation, acid gas removal techniques, CO₂ solvents such as amine, methanol and the like for absorption using a solvent, cryogenic freezing out of CO₂, and scrubbing with a basic solution. The separation step may also involve the separation of nitrogen. The separation step may involve two or more separation stages in series or in parallel. The separation stages may be of the same technique or may employ different techniques. For example, two PSA units in series may be employed. In one embodiment, a first PSA may be used to remove CO₂ while a second PSA may be used to remove O₂ and reject N₂. Separated oxygen may be returned to the secondary bioreactor. Another way to remove O₂ from the gas to be recycled back to the first bioreactor is the use of yet a third fermenter that gets supplied with microbes from the second fermenter. This third fermenter will not be sparged with O₂ therefore putting the microbes in O₂ starvation mode. If the off-gas of the second bioreactor is led through this third vessel, the microbes will take all remaining O₂ out such that the gas leaving this third reactor can now be returned to the first bioreactor. The microbes in that third vessel will be transferred back (returned, recycled) to the second bioreactor. There will be a constant passage and return of microbes from the second bioreactor to that third fermentation vessel.

In another embodiment, the secondary fermentation could be run in batch mode, to allow for cleaning/sterilization between batches, while the primary fermentation could be run continuously. Whether operated in a continuous or batch mode, fermentation broth from the second fermenter may be recycled back to the primary fermentation. This recycling can be beneficial to make use of unconsumed nutrients that were present in the secondary fermentation thereby reducing overall operating and nutrient costs.

### Fermentation and Separation

The term “fermentation” should be interpreted as a metabolic process that produces chemical changes in a substrate. For example, a fermentation process receives one or more substrates and produces one or more products through utilization of one or more microorganisms. The term “fermentation” should be interpreted as the process which receives one or more substrates and produces one or more products through the utilization of one or more microorganisms. Preferably the fermentation process includes the use of one or more bioreactors. The fermentation process may be described as either “batch” or “continuous”. “Batch fermentation” is used to describe a fermentation process where the bioreactor is filled with raw material, for example the carbon source, along with microorganisms, where the products remain in the bioreactor until fermentation is completed. In a “batch” process, after fermentation is completed, the products are extracted, and the bioreactor is cleaned before the next “batch” is started. “Continuous fermentation” is used to describe a fermentation process in which the fermentation process is extended for longer periods of time, and product and/or metabolite is extracted during fermentation. Preferably the fermentation process is continuous.

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Preferably the aqueous culture medium is a suitable microbial growth medium, such as a minimal microbial growth medium. Suitable media are well known in the art.

The culture/fermentation should desirably be carried out under appropriate conditions for production of the second stage product/final product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Target products (or second stage products) may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more second stage products from the broth. Alcohols and/or acetone may be recovered, for example, by distillation. Acids may be recovered, for example, by adsorption on activated charcoal. Separated microbial cells are preferably recycled back to the bioreactor. The cell-free permeate remaining after second stage products have been removed is also preferably returned to the bioreactor. Additional nutrients may be added to the cell-free permeate to replenish the medium before it is returned to the bioreactor.

In embodiment of the disclosure, prior to transferring first fermentation broth to the second bioreactor, a product of the first fermentation is removed from the first fermentation broth. The product removed may be some or all of the acetate. The product removed may be a secondary product of the fermentation process. Alternatively, a secondary product of the first fermentation process may be removed and recovered (or discarded) only after the second fermentation. So long as the secondary product is not further converted by the second microorganism and does not negatively affect the second fermentation, it need not be removed until after the second fermentation.

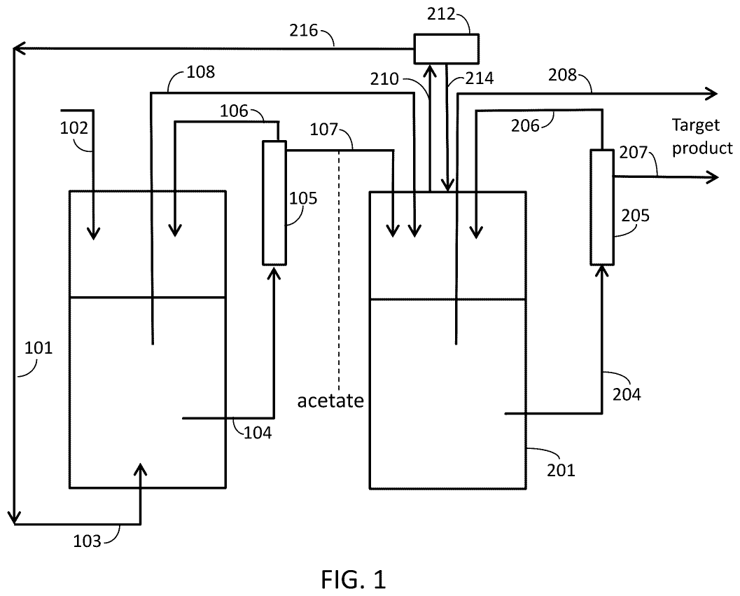

The methods and systems of the disclosure are herein described with reference to the Figures. FIG. 1 demonstrates a two-stage system to produce second stage product(s) from a gaseous stream comprising CO and H₂, or CO₂ and H₂. The system provides a primary bioreactor 101 having a media inlet 102, a gas inlet port 103, a separator means 104, a permeate stream outlet 107, and a bleed stream outlet 108. The primary bioreactor is connected to a secondary bioreactor 201, having a separator 205, a permeate stream outlet 207 and a bleed stream outlet 208.

In use, the primary bioreactor 101 may contain fermentation broth comprising a culture of one or more acetogenic bacteria in a liquid nutrient medium. Medium is added to the bioreactor 101 in a continuous or semi-continuous manner throughout the media inlet 102. A gaseous substrate is supplied to the bioreactor 101 via the gas inlet port 103. The separator means is adapted to receive at least a portion of broth from the bioreactor 101 via a first output conduit 104 and pass it through the separator 105 configured to substantially separate the microorganism cells (the retentate) from the rest of the fermentation broth (the permeate). At least a portion of the retentate is returned to the first bioreactor via a first return conduit 106, which ensures that the broth culture density is maintained at an optimal level. The separator 105 is adapted to pass at least a portion of the permeate out of the bioreactor 101 via a permeate delivery conduit 107. The permeate delivery conduit 107 feeds the cell free permeate to the secondary bioreactor 201. In certain embodiments of the disclosure, at least a portion of the cell free permeate is removed for product extraction and/or at least a portion of the cell free permeate is recycled to primary bioreactor with the remainder of the cell free permeate stream being fed to the secondary bioreactor 201. A broth bleed output 108 is provided to directly feed broth from the primary bioreactor 101 to the secondary bioreactor 202. In certain embodiments the broth bleed and permeate are combined prior to being fed to the secondary bioreactor.

Secondary bioreactor 201 contains a culture of one or more microorganisms in a liquid nutrient medium. An aerobic microorganism, such as yeast or E. coli, are used as specific examples, but any suitable microorganism may be employed in secondary bioreactor 201. Secondary bioreactor 201 receives broth and/or permeate from primary bioreactor 101 in a continuous or semi-continuous manner through broth bleed output 108 and permeate delivery conduit 107. The separator 205 is adapted to receive at least a portion of broth from secondary bioreactor 201 via a first output conduit 204. Separator 205 is configured to substantially separate the microorganism cells (the retentate) from the rest of the fermentation broth (the permeate). At least a portion of the retentate is returned to secondary bioreactor 201 via a second return conduit 206, which ensures that the broth culture density in secondary bioreactor 201 is maintained at an optimal level. Separator 205 is adapted to pass at least a portion of the permeate out of secondary bioreactor 201 via a permeate removal conduit 207. Broth bleed output 208 is provided directly to remove broth from secondary bioreactor 201. Broth bleed output 208 may be treated to remove the biomass for second stage product extraction using known methods. The substantially biomass free bleed stream and the permeate streams may be combined to produce a combined stream. In certain embodiments of the disclosure, the combined stream can be returned to the primary reactor to supplement the liquid nutrient medium being continuously added. In certain embodiments, it may be desirable to further process the recycle stream to remove undesired or desired by-products of the secondary fermentation. In certain embodiments, the pH of the recycle stream may be adjusted, and further vitamins and/or metals added to supplement the stream.

Tail gas stream 210 from secondary bioreactor 201 is passed to oxygen removal unit 212 to generate a substantially oxygen free CO₂ stream. Oxygen removal unit may be, for example, one or more PSA units, membrane separation units, scrubbers, absorption using solvent(s), or any combination thereof. Substantially oxygen free CO₂ stream is passed in line 216 and recycled to primary bioreactor 101 through inlet gas port 103. Substantially oxygen free means the stream comprises less than about 1 mol-percent O2, less than about 500 mol-ppm O₂ or less than about 100 mol-ppm O₂. O₂ that is removed from the tail gas 210 in oxygen removal unit 212 may be recycled to the secondary bioreactor 201 through line 214.

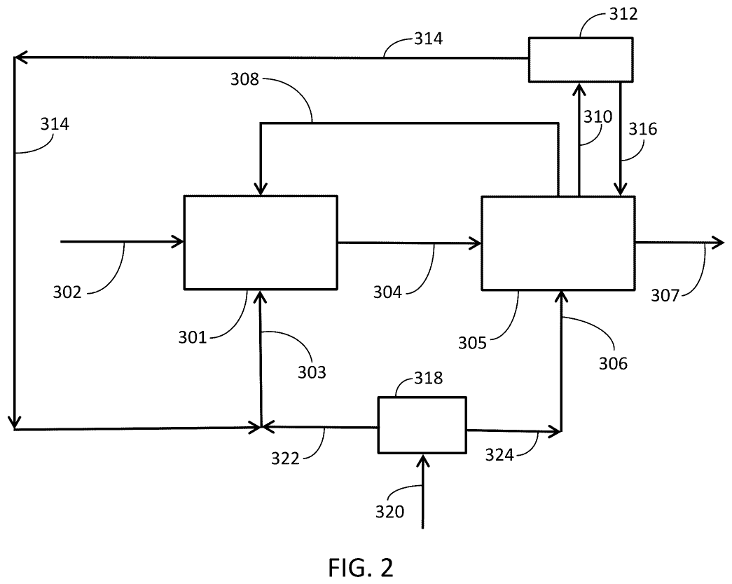

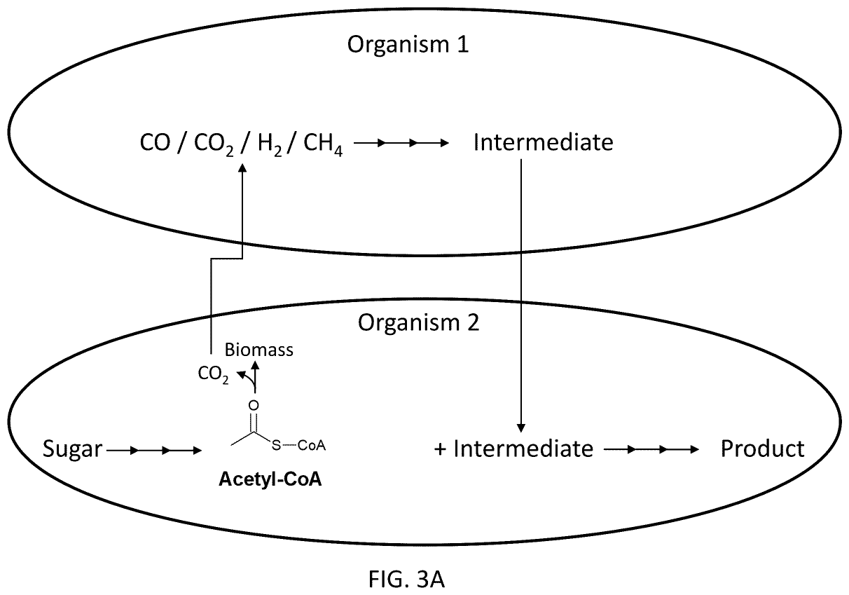

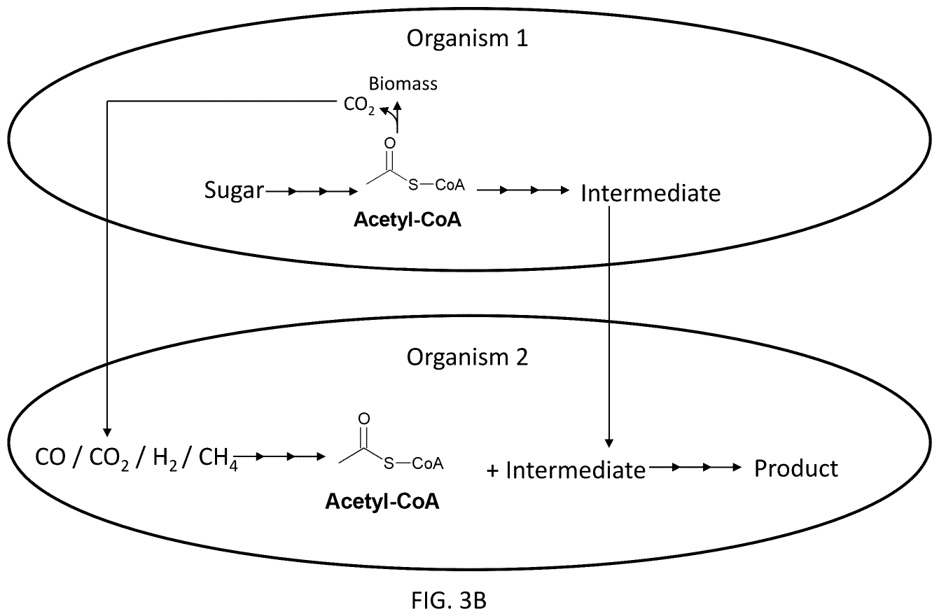

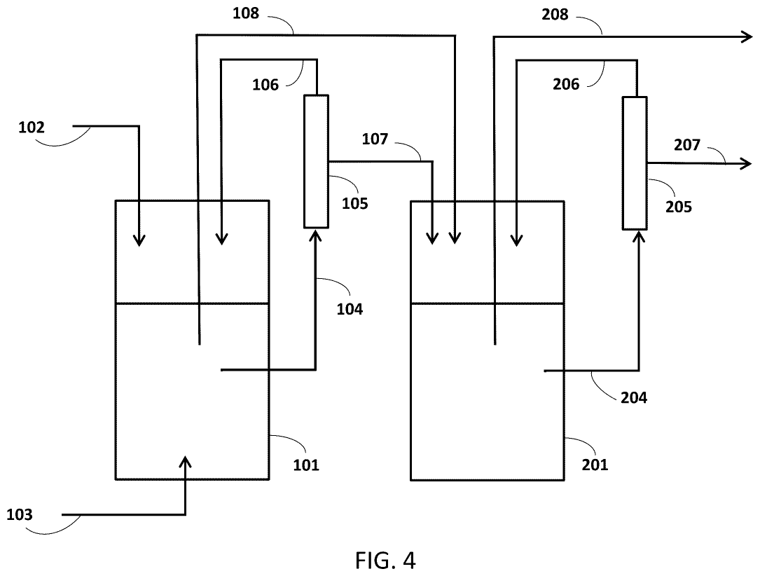

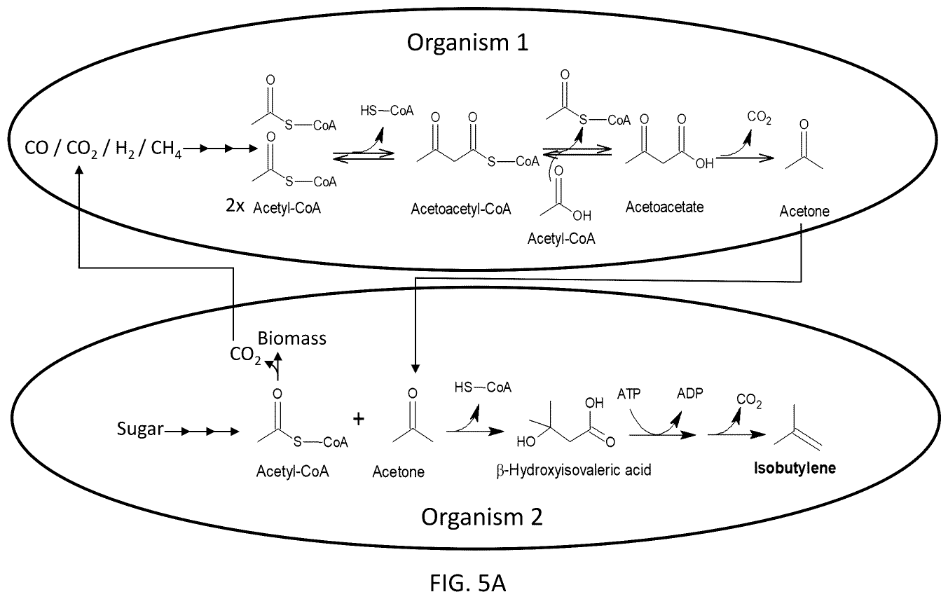

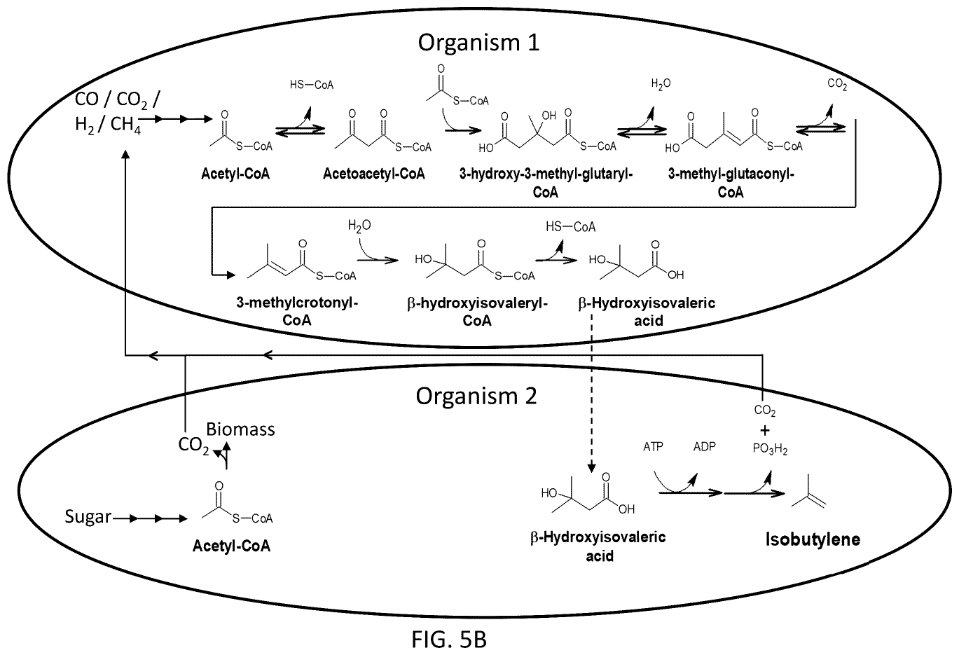

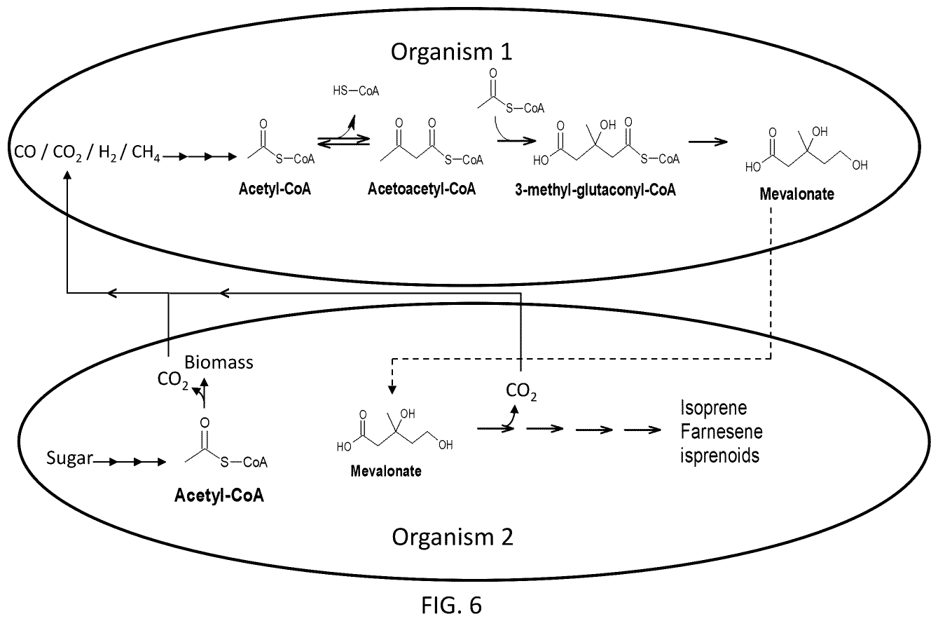

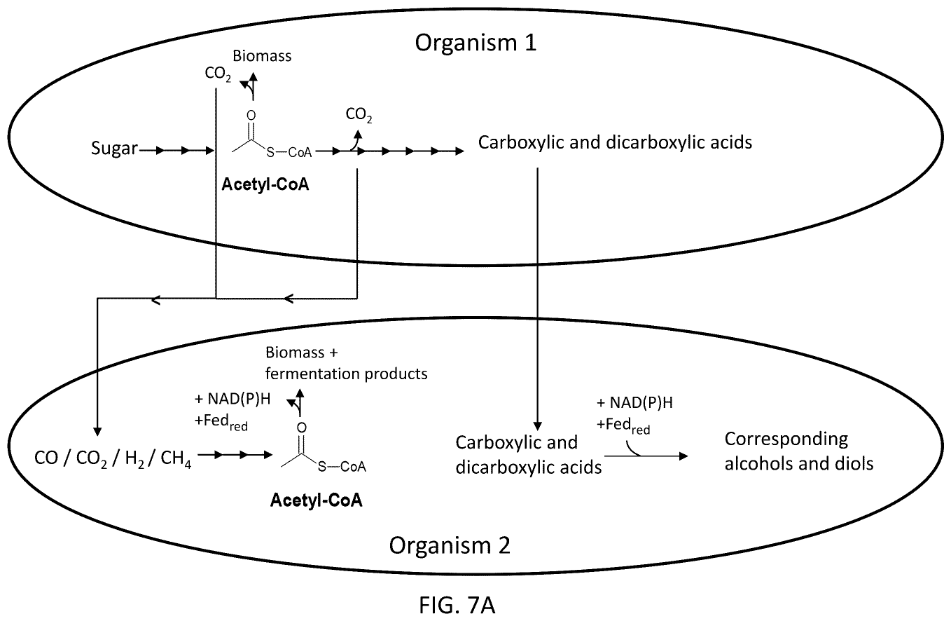

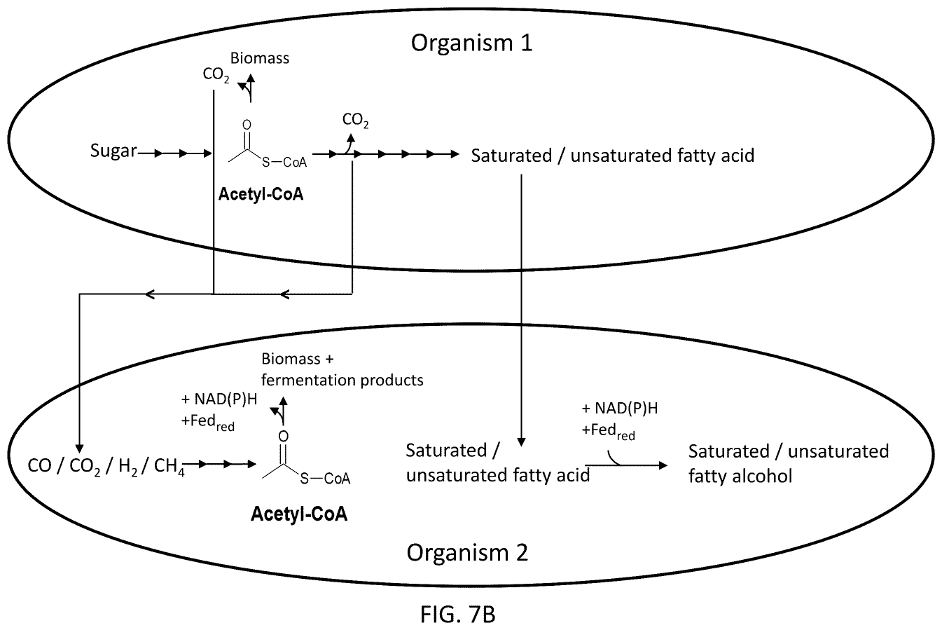

FIG. 2 demonstrates a simplified system for the production of second stage product(s) from a gaseous stream comprising CO and H₂, or CO₂ and H₂, wherein the substantially acetate-free media is recycled from the secondary bioreactor to the primary bioreactor. The system includes a primary anaerobic bioreactor 301 having a media inlet 302, a gas inlet port 303, an acetate-containing treated bleed stream 304, a secondary aerobic bioreactor 305, an oxygen source 306, a second stage product and biomass-containing product stream 307, and an acetate-depleted recycled media stream. In one embodiment, the gaseous stream is produced by an electrolyzer.

In use, the primary bioreactor 301 contains fermentation broth comprising a culture of one or more acetogenic bacteria in a liquid nutrient medium. Medium is added to primary bioreactor 301 through the media inlet 302. A gaseous substrate comprising either CO and optionally H₂, or CO₂ and H₂, or mixtures thereof, is supplied to the primary bioreactor 301 via a gas inlet port 303, wherein the gas is converted by the bacteria to acetate. The primary bioreactor 301 is maintained at a pH in the range of 2.5-5, or 3-4, or 6.5-7, with pH optionally partially controlled by the addition of base as necessary. The acetate product leaves the primary bioreactor in an aqueous broth stream, which is treated to remove the biomass using known methods. The resulting acetate-containing treated bleed stream 304 is fed to a secondary aerobic bioreactor 305. In the secondary bioreactor 305, acetate in the treated bleed stream is converted to second stage products and biomass by a microorganism, such as for example a microorganisms. Oxygen is supplied to the aerobic fermentation by an oxygen or air inlet port 306. The second stage product-containing microorganism cells are removed from the secondary bioreactor 305 by filtration, resulting in a second stage product- and biomass-containing product stream 307 and a permeate stream 308. Because the aerobic fermentation consumes acetate, the pH of the broth increases as acetic acid is consumed, and the pH of the permeate stream 308 is consequently nominally higher than the pH of the acetate-containing broth stream 304. The dilution rate of the secondary bioreactor 305 is maintained such that the pH of the permeate stream 308 remains in the range of, for example, 5-7; or 7.0-7.5; or 7.5-9; or 10-11. The acetate-depleted permeate stream 308 is returned to the primary bioreactor 301. In addition to recycling a substantial portion of the water, salts, metals, and other nutrients that make up the media of the primary bioreactor 301, the recycled permeate stream 308 acts to reduce significantly the cost of fermentation pH control relative to a system in which pH is controlled only by direct addition of base to the bioreactor media.

Tail gas generated in secondary bioreactor 305 is removed in tail gas line 310 and conducted to oxygen separation unit 312. Oxygen separation unit may be, for example, one or more PSA units, membrane separation units, scrubbers, absorption using solvent(s), or any combination thereof. At least oxygen is removed from the tail gas in the oxygen separation unit. The resulting CO₂ stream that is substantially free of O₂ is then conducted in line 314 to gas inlet port 303 and introduced into primary bioreactor 301. O₂ removed in the oxygen separation unit may be recycled to secondary bioreactor 305 through line 316. FIG. 2 further illustrates the embodiment employing the optional water electrolyzer. Water stream 320 is introduced into electrolyzer 318 where a H₂ stream is generated by electrolysis and the H₂ stream is conducted in line 322 to gas inlet port 303 and introduced into primary bioreactor 301 along with the gaseous substrate discussed above. Similarly, electrolyzer 318 generates an O2 stream, which is conducted in line 324 to inlet port 306 of secondary bioreactor 305.

In an embodiment, a method for the circular production of a commodity chemical is provided. By way of an example, circular production of tires, nylon, fabrics, textiles, bioplastics, polyhydroxyalkanoates (PHA), monoethylene glycol (MEG) etc. The method comprises converting used tires back to useful tires or tire components is carried out. According to one or more embodiments, used tires are thermally decomposed to form a gaseous stream. One or more components of this gaseous stream are then converted to butadiene monomer or tires. The method for the circular production of a commodity chemical comprises passing a gaseous substrate to a first bioreactor containing a culture of a recombinant C1-fixing microorganism capable of producing at least one first product in a culture medium such that the microorganism converts the gaseous substrate to the at least one first product; and recovering the at least one first product from the bioreactor; passing a gaseous substrate to a second bioreactor containing a culture of a recombinant C1-fixing microorganism capable of producing at least one second product selected from ethylene and olefins, or any combination thereof in a culture medium such that the microorganism converts the gaseous substrate to the at least one second product; and recovering the at least one second product from the bioreactor; polymerizing the at least one first product with the at least one second product in the presence of a suitable polymerization catalyst to form a hybrid polymer; and converting the hybrid polymer into a tire.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavor in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (that is, meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (for example, “or”) should be understood to mean either one, both, or any combination thereof of the alternatives. As used herein, the term “about” means±20% of the indicated range, value, or structure, unless otherwise indicated.

“A portion of” may refer to a volume or weight portion of a composition such as a fermentation broth or tail gas created in a fermentation process. The term may also refer to a subset of components of a composition, such as when a fermentation broth or tail gas has been subjected to a purification process to remove components—whether wanted or unwanted. Thus, if a composition comprises a mixture of components, the portion may comprise the same mixture of components, an altered ratio of components, or an altered set of components. “At least a portion” may include the entirety or all of a composition.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (for example, “such as”) provided herein, is intended merely to better illuminate the disclosure and does not pose a limitation on the scope of the invention unless otherwise claimed. No language in the specification should be construed as indicating any non-claimed element as essential to the practice of the disclosure.

Specific embodiments of this disclosure are described herein. Variations of those specific embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### Embodiments of the Disclosure

Embodiments of this disclosure are described herein. Variations of those embodiments may become apparent to those of ordinary skill in the art upon reading the foregoing description, and employment of such variations as appropriate, is intended to be within the scope as the disclosure may be practiced otherwise than as specifically described herein. Accordingly, this disclosure includes all modifications and equivalents of the subject matter recited in the claims as permitted by applicable law. Moreover, any combination of the above described elements in all possible variations thereof is encompassed by the disclosure unless otherwise indicated herein or otherwise clearly contradicted by context.

Embodiment 1: A method for producing at least one product from CO and optionally H₂, or from CO₂ and H₂, the method comprising:
a. introducing a gaseous substrate comprising at least CO and optionally H₂, or at least CO₂ and H₂ into a first-stage bioreactor containing a culture of at least one first-stage microorganism in a liquid nutrient medium and fermenting the gaseous substrate to produce one or more first-stage products;
b. treating the one or more first-stage products before introducing to a second-stage fermentation culture; and
c. introducing at least a portion of at least one of the first-stage products to a second-stage bioreactor containing a culture of at least one second-stage microorganism in a liquid nutrient medium and fermenting the first-stage product to produce one or more second-stage products.

Embodiment 2: The method of embodiment 1 wherein at least one of the one or more first-stage products and or at least one of the one or more second-stage products are incorporated into one or more articles or chemically converted into one or more final products.

Embodiment 3: The method of any of the embodiments 1 to 2 wherein the first-stage microorganism and the second-stage microorganism are each selected from yeast, fungi, or bacterium.

Embodiment 4: The method of any of the embodiments 1 to 3 wherein the one or more first-stage products comprise ethanol, acetate, or a mixture thereof.

Embodiment 5: The method of any of the embodiments 1 to 4 wherein the one or more second-stage products include alcohols, acids, biofuels, food products, food additives and ingredients, industrial enzymes, vitamins and nutritional supplements, antibiotics and pharmaceuticals, bioplastics, textiles and fibers, flavor and fragrance compounds, biochemicals and specialty chemicals, biogas and biomethane, biochemicals for agriculture, biopharmaceuticals, animal health products, biodegradable polymers, cellular agriculture products, amino acids, cellulosic biofuels, bio-based chemical intermediates, feed products, beverage products, medicinal products, silk proteins, cultured protein or mixture thereof.

Embodiment 6: The method of any of the embodiments 1 to 5 wherein the one or more second stage products include at least one alcohol and carbon dioxide, and further comprising recycling the carbon dioxide to the first-stage bioreactor.

Embodiment 7: The method of any of the embodiments 1 to 6, wherein one or more first-stage products is selected from ethanol, isopropanol, monoethylene glycol, sulfuric acid, propylene, sodium hydroxide, sodium carbonate, ammonia, benzene, acetic acid, acetate, ethylene, ethylene oxide, formaldehyde, methanol or any combination thereof.

Embodiment 8: The method of any of the embodiments 2 to 7, wherein one or more first-stage products, one or more second-stage products, one or more articles, and or one or more final products are selected from ethanol, acetic acid, butyric acid, propionic acid, methane, itaconic acid, 3-Hydroxypropionic acid, acrylic acid, vinyl acetate, malonic acid, acrylamide, propiolactone, oligomers, and polyesters, lactic acid, succinic acid, formic acid, mevalonic acid, tyrosine, phloroglucinol, fatty acids, lipids, β-Caryophyllene, polyhydroxyalkanoates, isobutyl acetate, hydrogen, monellin, diacylglycerides, a C₅-C₂₀ alcohol, an organic acid, a ketone, a lipid, propanol, butanol, 2,3-butanediol, 1,4-butanediol, 1,3-butanediol, butyrate, propionate, caproate, butadiene, isobutylene, polyhydroxyalkanoates, succinate, mevalonate, vinyl acetate, Vinyl Acetate Monomer (VAM), cellulose acetate, pigments, ethyl cellulose, food additives, pharmaceuticals, acetylcholine, acetyl coenzyme A, polyvinyl acetate (PVA) emulsions, acetic acid derivatives, acetate esters such as butyl acetate, ethyl acetate, methyl acetate, acetone, acetophenone, acetonitrile, acetamide, acetylene, diethyl ether (ethoxyethane), acetic ether (ethyl acetate dimer), acetic acid anhydride (ethanoic anhydride), acetyl salicylic acid (Aspirin), acetyl cellulose, acetic acid esters of glycerol (Acetins), ethylene vinyl acetate (EVA), acetate ions, acetyl bromide, sodium acetate, acetate buffer, acetylferrocene, benzyl acetate, acetate dyes, acetic acid aluminium salt, acetylated starch, vinyl acetate copolymer, ethyl acetate peroxide, ethyl acetate sulfate, acetyl glucosamine, propyl acetate, acetyl glycine, acetic acid ammonium salt, acetic acid ethylene ester (Ethylidene Diacetate), acetic acid citrate solution, acetylcholinesterase, acetylcholinesterase inhibitors, acetylacetonate, acetylthiocholine, acetylated wood, acetyl fluoride, acetic acid lithium salt, ethyl acetate nitrate, acetyl triethyl citrate acetylated lanolin, acetaldehyde, ethyl acetoacetate, ethyl lactate, acetated alcohol or any combination thereof.

Embodiment 9: The method of embodiment 2, wherein the one or more first products, one or more second products, or one or more articles is incorporated into or utilized as adhesives, water based paints, textile/paper coatings and films, textiles, cigarette filters, fabrics for clothing, upholstery and linens, packaging, pharmaceuticals, dyes and perfumes, solvent in paints, flavoring agent, thermoplastic material, eyeglass frames, various consumer goods, inks, coating material, binder, cosmetic, food, flavour enhancers, preservatives acidity regulator, solvent in production of pharmaceuticals, solvent, chemical manufacturing, plastic production, topical medications, astringent, food and pharmaceutical industry as stabilizer and thickening agent, initiator in the polymerization of some monomers, surfactant and emulsifying agent, solvent in coatings, paints and cleaning applications, cleaning products such as detergents, disinfectants, surface cleaners and sanitizers, biofuel, hand sanitizers, medicines, including liquid formulations, tinctures and syrups; extraction and dissolution of active ingredients, precursor or intermediate in the synthesis of various chemicals ex acetaldehyde, ethylene, ethyl acetate, acetic acid, and ethylene glycol, plastics, resins, fibers, surfactant and in the production of detergents, textiles and personal care products, resins, plastics, dyes and other chemicals, solvent in coatings, adhesives and automotive industries, extraction agent and in the production of pharmaceuticals and industrial chemicals, intermediate in synthesis of pharmaceuticals, cosmetics, dyes and flavors, biodegradable solvent used in paints coatings and personal care products, for the production of propiolactone, oligomers, and polyesters, manufacture clothing, linens, upholstery, and other textile products, food and beverages, cleaning products both for household and industrial cleaning agents, printing and photography, various industrial chemicals, fertilizer, superphosphates, detergents synthetic resins, dyestuffs, petroleum catalysts, insecticides, antifreeze, pigments, enamels, printing inks, coated fabrics, paper, cellophane, acetate textile, viscose textiles, lubricants, non-ferrous metals, batteries, anaesthetics, fruit ripening agents, fertilizers, safety glass, metal cutting gases, welding gases, high velocity thermal sprays, refrigerants, or any combination thereof.

Embodiment 10: The method of any of the embodiments 1 to 9, wherein the one or more first-stage products, one or more second-stage products, or any combination thereof is incorporated into or utilized as a personal care product, a fragrance, a cosmetic, or a home care product including perfume, cologne, aftershave, shampoo, shower gel, soap, body lotion, laundry detergents, fabric softeners, dryer sheets, room fresheners, carpet fresheners, essential oils, cleaning, polishing, scouring abrasives, toiletries, sanitary preparations, tissue, nail products, diapers, bandages, sunscreen, nourishing cream, hand cream, lipstick, lip gloss, sun oil, massage cream, cleansing cream, facial pack, serums for cosmetic purposes, cocoa butter, skin care product, retinol cream, muscle gel, body paint, cooling spray, cold cream, pomade, cleaning products, dishwashing liquid or any combination thereof.

Embodiment 11: The method of any of the embodiments 1 to 10, further comprising incorporating one or more first-stage products, one or more second-stage products, or any combination thereof as a commodity chemical into a product selected from ethanol, acetate, 1-butanol, butyrate, 2,3-butanediol, lactate, butene, butadiene, methyl ethyl ketone (2-butanone), ethylene, acetone, isopropanol, lipids, 3-hydroxyproprionate, terpenes, isoprene, fatty acids, 2-butanol, 1,2-propanediol, 1 propanol, 1 hexanol, 1 octanol, chorismate-derived products, 3-hydroxybutyrate, 1,3-butanediol, 1,4-butanediol, 2-hydroxyisobutyrate, 2-hydroxyisobutyric acid, isobutylene, adipic acid, 1,3-hexanediol, 3-methyl-2-butanol, 2-buten-1-ol, isovalerate, isoamyl alcohol, monoethylene glycol, antiseptic hand rubs, therapeutic treatments for methylene glycol poisoning, therapeutic treatments for methanol poisoning, pharmaceutical solvent for pain medication, oral hygiene products, antimicrobial preservative, engine fuel, rocket fuel, plastics, fuel cells, home fireplace fuels, industrial chemical precursor, cannabis solvent, winterization extraction solvent, paint masking product, paint, tincture, purification and extraction of DNA and RNA, cooling bath for various chemical reactions, ethylene to raw material, anaesthetic, ethylene and nitrogen in fruit ripening, fertilizer, safety glass, oxy-fuel in metal cutting, welding, high velocity thermal spraying, refrigerant, raw material to polyethylene, raw material to PET, raw material to PVC, fibers, packaging, coatings, adhesives, ethylene dichloride (EDC), vinyl chloride monomer (VCM), alpha olefins, linear alpha olefins, detergent alcohols, plasticizer alcohols, vinyl acetate monomer (VAM), barrier resins, industrial ethanol, ethyl acetate, ethyl acrylate, polyethylene oligomers, Ultra-high-molecular-weight polyethylene (UHMWPE), Ultra-low-molecular-weight polyethylene (ULMWPE or PE-WAX), High-molecular-weight polyethylene (HMWPE), High-density polyethylene (HDPE), High-density cross-linked polyethylene (HDXLPE), Cross-linked polyethylene (PEX or XLPE), Medium-density polyethylene (MDPE), Low-density polyethylene (LDPE), Very-low-density polyethylene (VLDPE), Chlorinated polyethylene (CPE), films, food packaging, non-food packaging, shrink film, stretch film, containers, drums, household goods, caps, pallets, pipes, refuse sacks, carrier bags, industrial lining, ethylene oxide, ethoxylates, shampoo, kitchen cleaners, glycol ethers, ethanolamines, surfactants, personal care products, pet care products, candles, car care products, polyester fibers, textiles, nonwovens, cover stock for diapers, road-building fabrics, filters, fiberfill, felts, transportation upholstery, paper reinforcement, tape reinforcement, tents, rope, cordage, sails, fish netting, seatbelts, laundry bags, synthetic artery replacements, carpets, rugs, apparel, sheets, pillowcases, towels, curtains, draperies, bed ticking, blankets, liquid coolant, antifreeze, preservative, dehydrating agent, drilling fluid, polyester resins, insulation materials, polyester film, de-icing fluid, heat transfer fluid, automotive antifreeze, water-based adhesives, latex paints, asphalt emulsions, electrolytic capacitors, synthetic leather, polyester resin PET, jars, bottles, plastic bottles, high-strength fibers, Dacron, durable-press blends, insulated clothing, furniture filling, pillow filling, artificial silk, carpet fiber, automobile tire yarns, conveyor belts, drive belts, reinforcement for fire and garden hoses, nonwoven fabrics for stabilizing drainage ditches, culverts, railroad beds, nonwovens for diaper topsheets, disposable medical garments, high-strength plastics, magnetic recording tape, photographic film, as feedstock, solvents for cosmetics, inks, medicinal tablets, disinfectants, sterilizers, skin creams, purification of vegetable oil and fats, purification of animal oil and fats, cleaning agent, drying agent, aerosol solvent, derivative ketones, isopropylamines, isopropyl esters, propylene, polypropylene oligomers, polymerization modifier, coupling agent, heat resistant articles, kettles, food containers, disposable bottles, clear bags, flooring, mats, adhesive stickers, foam polypropylene, building materials, hydrophilic clothing, medical dressings, or any combination thereof.

Embodiment 12: The method of any of the embodiments 1 to 11, further comprising incorporating one or more first-stage products, one or more second-stage products, or any combination thereof as a commodity chemical into an article or converting a commodity chemical into a product selected from humectants, filters, fire extinguishing sprinkler system, fuel for warming foods, heat transfer fluids, non-reacted component in formulation, deodorizing or air purifying, softening agent, arts/craft glue/paste, toys, children products, freezer gel pack, treating wood rot and fungus, preserving biological tissues and organs, alkyd type resins, resin esters, enamels, lacquers, latex paint, asphalt emulsion, thermoplastic resin, hydrate inhibition agent, agent for removing water vapor, shoe polish, vaccines, screen cleaning solution, water-based hydraulic fluid, heat transfer for liquid cooled computers, personal lubricant, lubricant, toothpaste, anti-foaming agent in food industrial applications, flame resistant hydraulic fluids, additive for electrolytic polishing belts, industrial solvent, trash bags, shower curtains, cups, utensils, medical devices, durable goods, nondurable goods, plastic sacks, plastic lids, industrial strapping, construction materials, felt, ovenable trays, frozen food trays, microwavable tray, artificial vascular scaffolds, vascular prostheses, woven devices, polyester-based prostheses, vehicle liner material, soaps, cosmetic products, laundry detergent jugs, laundry detergent, soap microplastic, microbeads, cosmetic product microbeads, detergent pods, disinfectant with scrubbing agents, toothpaste with microbeads, face wash, conditioner, body wash, hand cleaner, exfoliating products, bath products, shower gels, powder laundry detergent, lotions, deodorants, toilet cleaners, sunscreen, shopping bags, mouthwash bottles, peanut butter containers, salad dressing and vegetable oil containers, polar fleece fiber, tote bags, paneling, milk jugs, juice bottles, bleach bottles, motor oil bottles, cercal box liners, recycling containers, floor tile, drainage pipe, benches, picnic tables, fencing, wire jacketing, sliding windows, decks, mud flaps, roadway gutters, speed bumps, squeezable bottles, bread, dry cleaning bags, trash can liners, trash cans, compost bins, shipping envelopes, lumber, syrup bottles, ketchup bottles, straws, medicine bottles, battery cables, battery cases, disposable plates and cups, egg cartons, carry-out containers, compact disc cases, signs and displays, synthetic fibers, yarn, stable phase change material, thermal energy storage material, nylon 6,6, nylon, tires, rubber, adiponitrile, shoes, footwear, or any combination thereof.

Embodiment 13: The method of any of the embodiments 1 to 12, further comprising downstream processing of the one or more first-stage products, one or more second-stage products, or any combination thereof to obtain surfactants, greases, algae products, fuels, chemicals, or nutrition products.

Embodiment 14: A method for the circular production of a commodity chemical, the method comprising:
1) passing a gaseous substrate to a first stage fermentation containing a culture of a recombinant C1-fixing microorganism capable of producing at least one first product in a culture medium such that the microorganism converts the gaseous substrate to the at least one first product; and recovering the at least one first product from the first-stage fermentation;
2) passing the at least one first product to a second stage fermentation containing a culture of a recombinant C1-fixing microorganism capable of producing at least one second product selected from ethylene and olefins, or any combination thereof in a culture medium such that the microorganism converts the at least one first product to the at least one second product; and recovering the at least one second product from the second-stage fermentation; and
3) converting the at least one second product into an article.

Embodiment 15: A method for producing at least one product from C1 carbon source including methane, the method comprising:
a. introducing a gaseous substrate comprising at least CO and optionally H₂, or at least CO₂ and H₂ into a first-stage bioreactor containing a culture of at least one first-stage microorganism in a liquid nutrient medium and fermenting the gaseous substrate to produce one or more first-stage products; and
b. introducing at least a portion of at least one of the first-stage products to a second-stage bioreactor containing a culture of at least one second-stage microorganism in a liquid nutrient medium and fermenting the first-stage product to produce one or more second-stage products.

Embodiment 16: The method of embodiment 15, wherein at least one of the one or more first-stage products and or at least one of the one or more second-stage products are incorporated into one or more articles or chemically converted into one or more final products.

Embodiment 17: The method of any of the embodiments 15 to 16, wherein the first-stage microorganism and the second-stage microorganism are each selected from yeast, fungi, or bacterium.

Embodiment 18: The method of any of the embodiments 15 to 17, wherein one or more first-stage products, one or more second-stage products, one or more articles, and or one or more final products are selected from ethanol, acetic acid, butyric acid, propionic acid, methane, itaconic acid, 3-Hydroxypropionic acid, acrylic acid, vinyl acetate, malonic acid, acrylamide, propiolactone, oligomers, and polyesters, lactic acid, succinic acid, formic acid, mevalonic acid, tyrosine, phloroglucinol, fatty acids, lipids, β-Caryophyllene, polyhydroxyalkanoates, isobutyl acetate, hydrogen, monellin, diacylglycerides, a C5-C20 alcohol, an organic acid, a ketone, a lipid, propanol, butanol, 2,3-butanediol, 1,4-butanediol, 1,3-butanediol, butyrate, propionate, caproate, butadiene, isobutylene, polyhydroxyalkanoates, succinate, mevalonate, vinyl acetate, Vinyl Acetate Monomer (VAM), cellulose acetate, pigments, ethyl cellulose, food additives, pharmaceuticals, acetylcholine, acetyl coenzyme A, polyvinyl acetate (PVA) emulsions, acetic acid derivatives, acetate esters such as butyl acetate, ethyl acetate, methyl acetate, acetone, acetophenone, acetonitrile, acetamide, acetylene, diethyl ether (ethoxyethane), acetic ether (ethyl acetate dimer), acetic acid anhydride (ethanoic anhydride), acetyl salicylic acid (Aspirin), acetyl cellulose, acetic acid esters of glycerol (Acetins), ethylene vinyl acetate (EVA), acetate ions, acetyl bromide, sodium acetate, acetate buffer, acetylferrocene, benzyl acetate, acetate dyes, acetic acid aluminium salt, acetylated starch, vinyl acetate copolymer, ethyl acetate peroxide, ethyl acetate sulfate, acetyl glucosamine, propyl acetate, acetyl glycine, acetic acid ammonium salt, acetic acid ethylene ester (Ethylidene Diacetate), acetic acid citrate solution, acetylcholinesterase, acetylcholinesterase inhibitors, acetylacetonate, acetylthiocholine, acetylated wood, acetyl fluoride, acetic acid lithium salt, ethyl acetate nitrate, acetyl triethyl citrate acetylated lanolin, acetaldehyde, ethyl acetoacetate, ethyl lactate, acetated alcohol or any combination thereof.

Embodiment 19: The method of any of the embodiments 15 to 18, wherein the one or more first products, one or more second products, or one or more articles is incorporated into or utilized as adhesives, water based paints, textile/paper coatings and films, textiles, cigarette filters, fabrics for clothing, upholstery and linens, packaging, pharmaceuticals, dyes and perfumes, solvent in paints, flavoring agent, thermoplastic material, eyeglass frames, various consumer goods, inks, coating material, binder, cosmetic, food, flavour enhancers, preservatives acidity regulator, solvent in production of pharmaceuticals, solvent, chemical manufacturing, plastic production, topical medications, astringent, food and pharmaceutical industry as stabilizer and thickening agent, initiator in the polymerization of some monomers, surfactant and emulsifying agent, solvent in coatings, paints and cleaning applications, cleaning products such as detergents, disinfectants, surface cleaners and sanitizers, biofuel, hand sanitizers, medicines, including liquid formulations, tinctures and syrups; extraction and dissolution of active ingredients, precursor or intermediate in the synthesis of various chemicals ex acetaldehyde, ethylene, ethyl acetate, acetic acid, and ethylene glycol, plastics, resins, fibers, surfactant and in the production of detergents, textiles and personal care products, resins, plastics, dyes and other chemicals, solvent in coatings, adhesives and automotive industries, extraction agent and in the production of pharmaceuticals and industrial chemicals, intermediate in synthesis of pharmaceuticals, cosmetics, dyes and flavors, biodegradable solvent used in paints coatings and personal care products, for the production of propiolactone, oligomers, and polyesters, manufacture clothing, linens, upholstery, and other textile products, food and beverages, cleaning products both for household and industrial cleaning agents, printing and photography, various industrial chemicals, fertilizer, superphosphates, detergents synthetic resins, dyestuffs, petroleum catalysts, insecticides, antifreeze, pigments, enamels, printing inks, coated fabrics, paper, cellophane, acetate textile, viscose textiles, lubricants, non-ferrous metals, batteries, anaesthetics, fruit ripening agents, fertilizers, safety glass, metal cutting gases, welding gases, high velocity thermal sprays, refrigerants, or any combination thereof as a personal care product, a fragrance, a cosmetic, or a home care product including perfume, cologne, aftershave, shampoo, shower gel, soap, body lotion, laundry detergents, fabric softeners, dryer sheets, room fresheners, carpet fresheners, essential oils, cleaning, polishing, scouring abrasives, toiletries, sanitary preparations, tissue, nail products, diapers, bandages, sunscreen, nourishing cream, hand cream, lipstick, lip gloss, sun oil, massage cream, cleansing cream, facial pack, serums for cosmetic purposes, cocoa butter, skin care product, retinol cream, muscle gel, body paint, cooling spray, cold cream, pomade, cleaning products, dishwashing liquid or any combination thereof.

Embodiment 20: The method of any of the embodiments 15 to 19, further comprising incorporating one or more first-stage products, one or more second-stage products, or any combination thereof into a product selected from ethanol, acetate, 1-butanol, butyrate, 2,3-butanediol, lactate, butene, butadiene, methyl ethyl ketone (2-butanone), ethylene, acetone, isopropanol, lipids, 3-hydroxyproprionate, terpenes, isoprene, fatty acids, 2-butanol, 1,2-propanediol, 1 propanol, 1 hexanol, 1 octanol, chorismate-derived products, 3-hydroxybutyrate, 1,3-butanediol, 1,4-butanediol, 2-hydroxyisobutyrate, 2-hydroxyisobutyric acid, isobutylene, adipic acid, 1,3-hexanediol, 3-methyl-2-butanol, 2-buten-1-ol, isovalerate, isoamyl alcohol, monoethylene glycol, antiseptic hand rubs, therapeutic treatments for methylene glycol poisoning, therapeutic treatments for methanol poisoning, pharmaceutical solvent for pain medication, oral hygiene products, antimicrobial preservative, engine fuel, rocket fuel, plastics, fuel cells, home fireplace fuels, industrial chemical precursor, cannabis solvent, winterization extraction solvent, paint masking product, paint, tincture, purification and extraction of DNA and RNA, cooling bath for various chemical reactions, ethylene to raw material, anaesthetic, ethylene and nitrogen in fruit ripening, fertilizer, safety glass, oxy-fuel in metal cutting, welding, high velocity thermal spraying, refrigerant, raw material to polyethylene, raw material to PET, raw material to PVC, fibers, packaging, coatings, adhesives, ethylene dichloride (EDC), vinyl chloride monomer (VCM), alpha olefins, linear alpha olefins, detergent alcohols, plasticizer alcohols, vinyl acetate monomer (VAM), barrier resins, industrial ethanol, ethyl acetate, ethyl acrylate, polyethylene oligomers, Ultra-high-molecular-weight polyethylene (UHMWPE), Ultra-low-molecular-weight polyethylene (ULMWPE or PE-WAX), High-molecular-weight polyethylene (HMWPE), High-density polyethylene (HDPE), High-density cross-linked polyethylene (HDXLPE), Cross-linked polyethylene (PEX or XLPE), Medium-density polyethylene (MDPE), Low-density polyethylene (LDPE), Very-low-density polyethylene (VLDPE), Chlorinated polyethylene (CPE), films, food packaging, non-food packaging, shrink film, stretch film, containers, drums, household goods, caps, pallets, pipes, refuse sacks, carrier bags, industrial lining, ethylene oxide, ethoxylates, shampoo, kitchen cleaners, glycol ethers, ethanolamines, surfactants, personal care products, pet care products, candles, car care products, polyester fibers, textiles, nonwovens, cover stock for diapers, road-building fabrics, filters, fiberfill, felts, transportation upholstery, paper reinforcement, tape reinforcement, tents, rope, cordage, sails, fish netting, seatbelts, laundry bags, synthetic artery replacements, carpets, rugs, apparel, sheets, pillowcases, towels, curtains, draperies, bed ticking, blankets, liquid coolant, antifreeze, preservative, dehydrating agent, drilling fluid, polyester resins, insulation materials, polyester film, de-icing fluid, heat transfer fluid, automotive antifreeze, water-based adhesives, latex paints, asphalt emulsions, electrolytic capacitors, synthetic leather, polyester resin PET, jars, bottles, plastic bottles, high-strength fibers, Dacron, durable-press blends, insulated clothing, furniture filling, pillow filling, artificial silk, carpet fiber, automobile tire yarns, conveyor belts, drive belts, reinforcement for fire and garden hoses, nonwoven fabrics for stabilizing drainage ditches, culverts, railroad beds, nonwovens for diaper topsheets, disposable medical garments, high-strength plastics, magnetic recording tape, photographic film, as feedstock, solvents for cosmetics, inks, medicinal tablets, disinfectants, sterilizers, skin creams, purification of vegetable oil and fats, purification of animal oil and fats, cleaning agent, drying agent, aerosol solvent, derivative ketones, isopropylamines, isopropyl esters, propylene, polypropylene oligomers, polymerization modifier, coupling agent, heat resistant articles, kettles, food containers, disposable bottles, clear bags, flooring, mats, adhesive stickers, foam polypropylene, building materials, hydrophilic clothing, medical dressings, or any combination thereof.

Embodiment 21: A method for the circular production of a commodity chemical, the method comprising:
1) passing a gaseous substrate to a first bioreactor containing a culture of a recombinant C1-fixing microorganism capable of producing at least one first product in a culture medium such that the microorganism converts the gaseous substrate to the at least one first product; and recovering the at least one first product from the bioreactor;
2) passing a gaseous substrate to a second bioreactor containing a culture of a recombinant C1-fixing microorganism capable of producing at least one second product selected from ethylene and olefins, or any combination thereof in a culture medium such that the microorganism converts the gaseous substrate to the at least one second product; and recovering the at least one second product from the bioreactor;
3) polymerizing the at least one first product with the at least one second product in the presence of a suitable polymerization catalyst to form a hybrid polymer;
4) converting the hybrid polymer into a tire; and
5) converting a used tire into the gaseous substrate for a bioreactor selected from the first bioreactor, the second bioreactor, or any combination thereof.

1. A method for producing at least one product from CO and optionally H₂, or from CO₂ and H₂, the method comprising:
a. introducing a gaseous substrate comprising at least CO and optionally H₂, or at least CO₂ and H₂ into a first-stage bioreactor containing a culture of at least one first-stage microorganism in a liquid nutrient medium and fermenting the gaseous substrate to produce one or more first-stage products;
b. treating the one or more first-stage products before introducing to a second-stage fermentation culture; and
c. introducing at least a portion of at least one of the first-stage products to a second-stage bioreactor containing a culture of at least one second-stage microorganism in a liquid nutrient medium and fermenting the first-stage product to produce one or more second-stage products.

2. The method of claim 1 wherein at least one of the one or more first-stage products and or at least one of the one or more second-stage products are incorporated into one or more articles or chemically converted into one or more final products.

3. The method of claim 1 wherein the first-stage microorganism and the second-stage microorganism are each selected from yeast, fungi, or bacterium.

4. The method of claim 1 wherein the one or more first-stage products comprise ethanol, acetate, or a mixture thereof.

5. The method of claim 1 wherein the one or more second-stage products include alcohols, acids, biofuels, food products, food additives and ingredients, industrial enzymes, vitamins and nutritional supplements, antibiotics and pharmaceuticals, bioplastics, textiles and fibers, flavor and fragrance compounds, biochemicals and specialty chemicals, biogas and biomethane, biochemicals for agriculture, biopharmaceuticals, animal health products, biodegradable polymers, cellular agriculture products, amino acids, cellulosic biofuels, bio-based chemical intermediates, feed products, beverage products, medicinal products, silk proteins, cultured protein or mixture thereof.

6. The method of claim 1 wherein the one or more second stage products include at least one alcohol and carbon dioxide, and further comprising recycling the carbon dioxide to the first-stage bioreactor.

7. The method of claim 1, wherein one or more first-stage products is selected from ethanol, isopropanol, monoethylene glycol, sulfuric acid, propylene, sodium hydroxide, sodium carbonate, ammonia, benzene, acetic acid, acetate, ethylene, ethylene oxide, formaldehyde, methanol or any combination thereof.

8. The method of claim 2, wherein one or more first-stage products, one or more second-stage products, one or more articles, and or one or more final products are selected from ethanol, acetic acid, butyric acid, propionic acid, methane, itaconic acid, 3-Hydroxypropionic acid, acrylic acid, vinyl acetate, malonic acid, acrylamide, propiolactone, oligomers, and polyesters, lactic acid, succinic acid, formic acid, mevalonic acid, tyrosine, phloroglucinol, fatty acids, lipids, β-Caryophyllene, polyhydroxyalkanoates, isobutyl acetate, hydrogen, monellin, diacylglycerides, a C₅-C₂₀ alcohol, an organic acid, a ketone, a lipid, propanol, butanol, 2,3-butanediol, 1,4-butanediol, 1,3-butanediol, butyrate, propionate, caproate, butadiene, isobutylene, polyhydroxyalkanoates, succinate, mevalonate, vinyl acetate, Vinyl Acetate Monomer (VAM), cellulose acetate, acetate fibers, acetic anhydride, acetaminophen, methyl acetate, vinegar, acetate plastic, dyes, pigments, ethyl cellulose, food additives, pharmaceuticals, acetylcholine, acetyl coenzyme A, polyvinyl acetate (PVA) emulsions, acetic acid derivatives, acetate esters such as butyl acetate, ethyl acetate, methyl acetate, acetone, acetophenone, acetonitrile, acetamide, acetylene, diethyl ether (ethoxyethane), acetic ether (ethyl acetate dimer), acetic acid anhydride (ethanoic anhydride), acetyl salicylic acid (Aspirin), acetyl cellulose, acetic acid esters of glycerol (Acetins), ethylene vinyl acetate (EVA), acetate ions, acetyl bromide, sodium acetate, acetate buffer, acetylferrocene, benzyl acetate, acetate dyes, acetic acid aluminium salt, acetylated starch, vinyl acetate copolymer, ethyl acetate peroxide, ethyl acetate sulfate, acetyl glucosamine, propyl acetate, acetyl glycine, acetic acid ammonium salt, acetic acid ethylene ester (Ethylidene Diacetate), acetic acid citrate solution, acetylcholinesterase, acetylcholinesterase inhibitors, acetylacetonate, acetylthiocholine, acetylated wood, acetyl fluoride, acetic acid lithium salt, ethyl acetate nitrate, acetyl triethyl citrate acetylated lanolin, acetaldehyde, ethyl acetoacetate, ethyl lactate, acetated alcohol or any combination thereof.

9. The method of claim 2, wherein the one or more first products, one or more second products, or one or more articles is incorporated into or utilized as adhesives, water based paints, textile/paper coatings and films, textiles, cigarette filters, fabrics for clothing, upholstery and linens, packaging, pharmaceuticals, dyes and perfumes, solvent in paints, flavoring agent, thermoplastic material, eyeglass frames, various consumer goods, inks, coating material, binder, cosmetic, food, flavour enhancers, preservatives acidity regulator, solvent in production of pharmaceuticals, solvent, chemical manufacturing, plastic production, topical medications, astringent, food and pharmaceutical industry as stabilizer and thickening agent, initiator in the polymerization of some monomers, surfactant and emulsifying agent, solvent in coatings, paints and cleaning applications, cleaning products such as detergents, disinfectants, surface cleaners and sanitizers, biofuel, hand sanitizers, medicines, including liquid formulations, tinctures and syrups; extraction and dissolution of active ingredients, precursor or intermediate in the synthesis of various chemicals ex acetaldehyde, ethylene, ethyl acetate, acetic acid, and ethylene glycol, plastics, resins, fibers, surfactant and in the production of detergents, textiles and personal care products, resins, plastics, dyes and other chemicals, solvent in coatings, adhesives and automotive industries, extraction agent and in the production of pharmaceuticals and industrial chemicals, intermediate in synthesis of pharmaceuticals, cosmetics, dyes and flavors, biodegradable solvent used in paints coatings and personal care products, for the production of propiolactone, oligomers, and polyesters, manufacture clothing, linens, upholstery, and other textile products, food and beverages, cleaning products both for household and industrial cleaning agents, printing and photography, various industrial chemicals, fertilizer, superphosphates, detergents synthetic resins, dyestuffs, petroleum catalysts, insecticides, antifreeze, pigments, enamels, printing inks, coated fabrics, paper, cellophane, acetate textile, viscose textiles, lubricants, non-ferrous metals, batteries, anaesthetics, fruit ripening agents, fertilizers, safety glass, metal cutting gases, welding gases, high velocity thermal sprays, refrigerants, or any combination thereof.

10. The method of claim 1, wherein the one or more first-stage products, one or more second-stage products, or any combination thereof is incorporated into or utilized as a personal care product, a fragrance, a cosmetic, or a home care product including perfume, cologne, aftershave, shampoo, shower gel, soap, body lotion, laundry detergents, fabric softeners, dryer sheets, room fresheners, carpet fresheners, essential oils, cleaning, polishing, scouring abrasives, toiletries, sanitary preparations, tissue, nail products, diapers, bandages, sunscreen, nourishing cream, hand cream, lipstick, lip gloss, sun oil, massage cream, cleansing cream, facial pack, serums for cosmetic purposes, cocoa butter, skin care product, retinol cream, muscle gel, body paint, cooling spray, cold cream, pomade, cleaning products, dishwashing liquid or any combination thereof.

11. The method of claim 1, further comprising incorporating one or more first-stage products, one or more second-stage products, or any combination thereof as a commodity chemical into a product selected from ethanol, acetate, 1-butanol, butyrate, 2,3-butanediol, lactate, butene, butadiene, methyl ethyl ketone (2-butanone), ethylene, acetone, isopropanol, lipids, 3-hydroxyproprionate, terpenes, isoprene, fatty acids, 2-butanol, 1,2-propanediol, 1 propanol, 1 hexanol, 1 octanol, chorismate-derived products, 3-hydroxybutyrate, 1,3-butanediol, 1,4-butanediol, 2-hydroxyisobutyrate, 2-hydroxyisobutyric acid, isobutylene, adipic acid, 1,3-hexanediol, 3-methyl-2-butanol, 2-buten-1-ol, isovalerate, isoamyl alcohol, monoethylene glycol, antiseptic hand rubs, therapeutic treatments for methylene glycol poisoning, therapeutic treatments for methanol poisoning, pharmaceutical solvent for pain medication, oral hygiene products, antimicrobial preservative, engine fuel, rocket fuel, plastics, fuel cells, home fireplace fuels, industrial chemical precursor, cannabis solvent, winterization extraction solvent, paint masking product, paint, tincture, purification and extraction of DNA and RNA, cooling bath for various chemical reactions, ethylene to raw material, anaesthetic, ethylene and nitrogen in fruit ripening, fertilizer, safety glass, oxy-fuel in metal cutting, welding, high velocity thermal spraying, refrigerant, raw material to polyethylene, raw material to PET, raw material to PVC, fibers, packaging, coatings, adhesives, ethylene dichloride (EDC), vinyl chloride monomer (VCM), alpha olefins, linear alpha olefins, detergent alcohols, plasticizer alcohols, vinyl acetate monomer (VAM), barrier resins, industrial ethanol, ethyl acetate, ethyl acrylate, polyethylene oligomers, Ultra-high-molecular-weight polyethylene (UHMWPE), Ultra-low-molecular-weight polyethylene (ULMWPE or PE-WAX), High-molecular-weight polyethylene (HMWPE), High-density polyethylene (HDPE), High-density cross-linked polyethylene (HDXLPE), Cross-linked polyethylene (PEX or XLPE), Medium-density polyethylene (MDPE), Low-density polyethylene (LDPE), Very-low-density polyethylene (VLDPE), Chlorinated polyethylene (CPE), films, food packaging, non-food packaging, shrink film, stretch film, containers, drums, household goods, caps, pallets, pipes, refuse sacks, carrier bags, industrial lining, ethylene oxide, ethoxylates, shampoo, kitchen cleaners, glycol ethers, ethanolamines, surfactants, personal care products, pet care products, candles, car care products, polyester fibers, textiles, nonwovens, cover stock for diapers, road-building fabrics, filters, fiberfill, felts, transportation upholstery, paper reinforcement, tape reinforcement, tents, rope, cordage, sails, fish netting, seatbelts, laundry bags, synthetic artery replacements, carpets, rugs, apparel, sheets, pillowcases, towels, curtains, draperies, bed ticking, blankets, liquid coolant, antifreeze, preservative, dehydrating agent, drilling fluid, polyester resins, insulation materials, polyester film, de-icing fluid, heat transfer fluid, automotive antifreeze, water-based adhesives, latex paints, asphalt emulsions, electrolytic capacitors, synthetic leather, polyester resin PET, jars, bottles, plastic bottles, high-strength fibers, Dacron, durable-press blends, insulated clothing, furniture filling, pillow filling, artificial silk, carpet fiber, automobile tire yarns, conveyor belts, drive belts, reinforcement for fire and garden hoses, nonwoven fabrics for stabilizing drainage ditches, culverts, railroad beds, nonwovens for diaper topsheets, disposable medical garments, high-strength plastics, magnetic recording tape, photographic film, as feedstock, solvents for cosmetics, inks, medicinal tablets, disinfectants, sterilizers, skin creams, purification of vegetable oil and fats, purification of animal oil and fats, cleaning agent, drying agent, aerosol solvent, derivative ketones, isopropylamines, isopropyl esters, propylene, polypropylene oligomers, polymerization modifier, coupling agent, heat resistant articles, kettles, food containers, disposable bottles, clear bags, flooring, mats, adhesive stickers, foam polypropylene, building materials, hydrophilic clothing, medical dressings, or any combination thereof.

12. The method of claim 1, further comprising incorporating one or more first-stage products, one or more second-stage products, or any combination thereof as a commodity chemical into an article or converting a commodity chemical into a product selected from humectants, filters, fire extinguishing sprinkler system, fuel for warming foods, heat transfer fluids, non-reacted component in formulation, deodorizing or air purifying, softening agent, arts/craft glue/paste, toys, children products, freezer gel pack, treating wood rot and fungus, preserving biological tissues and organs, alkyd type resins, resin esters, enamels, lacquers, latex paint, asphalt emulsion, thermoplastic resin, hydrate inhibition agent, agent for removing water vapor, shoe polish, vaccines, screen cleaning solution, water-based hydraulic fluid, heat transfer for liquid cooled computers, personal lubricant, lubricant, toothpaste, anti-foaming agent in food industrial applications, flame resistant hydraulic fluids, additive for electrolytic polishing belts, industrial solvent, trash bags, shower curtains, cups, utensils, medical devices, durable goods, nondurable goods, plastic sacks, plastic lids, industrial strapping, construction materials, felt, ovenable trays, frozen food trays, microwavable tray, artificial vascular scaffolds, vascular prostheses, woven devices, polyester-based prostheses, vehicle liner material, soaps, cosmetic products, laundry detergent jugs, laundry detergent, soap microplastic, microbeads, cosmetic product microbeads, detergent pods, disinfectant with scrubbing agents, toothpaste with microbeads, face wash, conditioner, body wash, hand cleaner, exfoliating products, bath products, shower gels, powder laundry detergent, lotions, deodorants, toilet cleaners, sunscreen, shopping bags, mouthwash bottles, peanut butter containers, salad dressing and vegetable oil containers, polar fleece fiber, tote bags, paneling, milk jugs, juice bottles, bleach bottles, motor oil bottles, cereal box liners, recycling containers, floor tile, drainage pipe, benches, picnic tables, fencing, wire jacketing, sliding windows, decks, mud flaps, roadway gutters, speed bumps, squeezable bottles, bread, dry cleaning bags, trash can liners, trash cans, compost bins, shipping envelopes, lumber, syrup bottles, ketchup bottles, straws, medicine bottles, battery cables, battery cases, disposable plates and cups, egg cartons, carry-out containers, compact disc cases, signs and displays, synthetic fibers, yarn, stable phase change material, thermal energy storage material, nylon 6,6, nylon, tires, rubber, adiponitrile, shoes, footwear, or any combination thereof.

13. The method of claim 1, further comprising downstream processing of the one or more first-stage products, one or more second-stage products, or any combination thereof to obtain surfactants, greases, algae products, fuels, chemicals, or nutrition products.

14. A method for the circular production of a commodity chemical, the method comprising:
1) passing a gaseous substrate to a first stage fermentation containing a culture of a recombinant C1-fixing microorganism capable of producing at least one first product in a culture medium such that the microorganism converts the gaseous substrate to the at least one first product; and recovering the at least one first product from the first-stage fermentation;
2) passing the at least one first product to a second stage fermentation containing a culture of a recombinant C1-fixing microorganism capable of producing at least one second product selected from ethylene and olefins, or any combination thereof in a culture medium such that the microorganism converts the at least one first product to the at least one second product; and recovering the at least one second product from the second-stage fermentation;
and
3) converting the at least one second product into an article.

15. A method for producing at least one product from a C1 carbon source including methane, the method comprising:
a. introducing a gaseous substrate comprising at least CO and optionally H₂, or at least CO₂ and H₂ into a first-stage bioreactor containing a culture of at least one first-stage microorganism in a liquid nutrient medium and fermenting the gaseous substrate to produce one or more first-stage products; and
b. introducing at least a portion of at least one of the first-stage products to a second-stage bioreactor containing a culture of at least one second-stage microorganism in a liquid nutrient medium and fermenting the first-stage product to produce one or more second-stage products.

16. The method of claim 15, wherein at least one of the one or more first-stage products and or at least one of the one or more second-stage products are incorporated into one or more articles or chemically converted into one or more final products.

17. The method of claim 15, wherein the first-stage microorganism and the second-stage microorganism are each selected from yeast, fungi, or bacterium.

18. The method of claim 15, wherein one or more first-stage products, one or more second-stage products, one or more articles, and or one or more final products are selected from ethanol, acetic acid, butyric acid, propionic acid, methane, itaconic acid, 3-Hydroxypropionic acid, acrylic acid, vinyl acetate, malonic acid, acrylamide, propiolactone, oligomers, and polyesters, lactic acid, succinic acid, formic acid, mevalonic acid, tyrosine, phloroglucinol, fatty acids, lipids, β-Caryophyllene, polyhydroxyalkanoates, isobutyl acetate, hydrogen, monellin, diacylglycerides, a C₅-C₂₀ alcohol, an organic acid, a ketone, a lipid, propanol, butanol, 2,3-butanediol, 1,4-butanediol, 1,3-butanediol, butyrate, propionate, caproate, butadiene, isobutylene, polyhydroxyalkanoates, succinate, mevalonate, vinyl acetate, Vinyl Acetate Monomer (VAM), cellulose acetate, acetate fibers, acetic anhydride, acetaminophen, methyl acetate, vinegar, acetate plastic, dyes, pigments, ethyl cellulose, food additives, pharmaceuticals, acetylcholine, acetyl coenzyme A, polyvinyl acetate (PVA) emulsions, acetic acid derivatives, acetate esters such as butyl acetate, ethyl acetate, methyl acetate, acetone, acetophenone, acetonitrile, acetamide, acetylene, diethyl ether (ethoxyethane), acetic ether (ethyl acetate dimer), acetic acid anhydride (ethanoic anhydride), acetyl salicylic acid (Aspirin), acetyl cellulose, acetic acid esters of glycerol (Acetins), ethylene vinyl acetate (EVA), acetate ions, acetyl bromide, sodium acetate, acetate buffer, acetylferrocene, benzyl acetate, acetate dyes, acetic acid aluminium salt, acetylated starch, vinyl acetate copolymer, ethyl acetate peroxide, ethyl acetate sulfate, acetyl glucosamine, propyl acetate, acetyl glycine, acetic acid ammonium salt, acetic acid ethylene ester (Ethylidene Diacetate), acetic acid citrate solution, acetylcholinesterase, acetylcholinesterase inhibitors, acetylacetonate, acetylthiocholine, acetylated wood, acetyl fluoride, acetic acid lithium salt, ethyl acetate nitrate, acetyl triethyl citrate acetylated lanolin, acetaldehyde, ethyl acetoacetate, ethyl lactate, acetated alcohol or any combination thereof.

19. The method of claim 15, wherein the one or more first products, one or more second products, or one or more articles is incorporated into or utilized as adhesives, water based paints, textile/paper coatings and films, textiles, cigarette filters, fabrics for clothing, upholstery and linens, packaging, pharmaceuticals, dyes and perfumes, solvent in paints, flavoring agent, thermoplastic material, eyeglass frames, various consumer goods, inks, coating material, binder, cosmetic, food, flavour enhancers, preservatives acidity regulator, solvent in production of pharmaceuticals, solvent, chemical manufacturing, plastic production, topical medications, astringent, food and pharmaceutical industry as stabilizer and thickening agent, initiator in the polymerization of some monomers, surfactant and emulsifying agent, solvent in coatings, paints and cleaning applications, cleaning products such as detergents, disinfectants, surface cleaners and sanitizers, biofuel, hand sanitizers, medicines, including liquid formulations, tinctures and syrups; extraction and dissolution of active ingredients, precursor or intermediate in the synthesis of various chemicals ex acetaldehyde, ethylene, ethyl acetate, acetic acid, and ethylene glycol, plastics, resins, fibers, surfactant and in the production of detergents, textiles and personal care products, resins, plastics, dyes and other chemicals, solvent in coatings, adhesives and automotive industries, extraction agent and in the production of pharmaceuticals and industrial chemicals, intermediate in synthesis of pharmaceuticals, cosmetics, dyes and flavors, biodegradable solvent used in paints coatings and personal care products, for the production of propiolactone, oligomers, and polyesters, manufacture clothing, linens, upholstery, and other textile products, food and beverages, cleaning products both for household and industrial cleaning agents, printing and photography, various industrial chemicals, fertilizer, superphosphates, detergents synthetic resins, dyestuffs, petroleum catalysts, insecticides, antifreeze, pigments, enamels, printing inks, coated fabrics, paper, cellophane, acetate textile, viscose textiles, lubricants, non-ferrous metals, batteries, anaesthetics, fruit ripening agents, fertilizers, safety glass, metal cutting gases, welding gases, high velocity thermal sprays, refrigerants, or any combination thereof as a personal care product, a fragrance, a cosmetic, or a home care product including perfume, cologne, aftershave, shampoo, shower gel, soap, body lotion, laundry detergents, fabric softeners, dryer sheets, room fresheners, carpet fresheners, essential oils, cleaning, polishing, scouring abrasives, toiletries, sanitary preparations, tissue, nail products, diapers, bandages, sunscreen, nourishing cream, hand cream, lipstick, lip gloss, sun oil, massage cream, cleansing cream, facial pack, serums for cosmetic purposes, cocoa butter, skin care product, retinol cream, muscle gel, body paint, cooling spray, cold cream, pomade, cleaning products, dishwashing liquid or any combination thereof.

20. The method of claim 15, further comprising incorporating one or more first-stage products, one or more second-stage products, or any combination thereof into a product selected from ethanol, acetate, 1-butanol, butyrate, 2,3-butanediol, lactate, butene, butadiene, methyl ethyl ketone (2-butanone), ethylene, acetone, isopropanol, lipids, 3-hydroxyproprionate, terpenes, isoprene, fatty acids, 2-butanol, 1,2-propanediol, 1 propanol, 1 hexanol, 1 octanol, chorismate-derived products, 3-hydroxybutyrate, 1,3-butanediol, 1,4-butanediol, 2-hydroxyisobutyrate, 2-hydroxyisobutyric acid, isobutylene, adipic acid, 1,3-hexanediol, 3-methyl-2-butanol, 2-buten-1-ol, isovalerate, isoamyl alcohol, monoethylene glycol, antiseptic hand rubs, therapeutic treatments for methylene glycol poisoning, therapeutic treatments for methanol poisoning, pharmaceutical solvent for pain medication, oral hygiene products, antimicrobial preservative, engine fuel, rocket fuel, plastics, fuel cells, home fireplace fuels, industrial chemical precursor, cannabis solvent, winterization extraction solvent, paint masking product, paint, tincture, purification and extraction of DNA and RNA, cooling bath for various chemical reactions, ethylene to raw material, anaesthetic, ethylene and nitrogen in fruit ripening, fertilizer, safety glass, oxy-fuel in metal cutting, welding, high velocity thermal spraying, refrigerant, raw material to polyethylene, raw material to PET, raw material to PVC, fibers, packaging, coatings, adhesives, ethylene dichloride (EDC), vinyl chloride monomer (VCM), alpha olefins, linear alpha olefins, detergent alcohols, plasticizer alcohols, vinyl acetate monomer (VAM), barrier resins, industrial ethanol, ethyl acetate, ethyl acrylate, polyethylene oligomers, Ultra-high-molecular-weight polyethylene (UHMWPE), Ultra-low-molecular-weight polyethylene (ULMWPE or PE-WAX), High-molecular-weight polyethylene (HMWPE), High-density polyethylene (HDPE), High-density cross-linked polyethylene (HDXLPE), Cross-linked polyethylene (PEX or XLPE), Medium-density polyethylene (MDPE), Low-density polyethylene (LDPE), Very-low-density polyethylene (VLDPE), Chlorinated polyethylene (CPE), films, food packaging, non-food packaging, shrink film, stretch film, containers, drums, household goods, caps, pallets, pipes, refuse sacks, carrier bags, industrial lining, ethylene oxide, ethoxylates, shampoo, kitchen cleaners, glycol ethers, ethanolamines, surfactants, personal care products, pet care products, candles, car care products, polyester fibers, textiles, nonwovens, cover stock for diapers, road-building fabrics, filters, fiberfill, felts, transportation upholstery, paper reinforcement, tape reinforcement, tents, rope, cordage, sails, fish netting, seatbelts, laundry bags, synthetic artery replacements, carpets, rugs, apparel, sheets, pillowcases, towels, curtains, draperies, bed ticking, blankets, liquid coolant, antifreeze, preservative, dehydrating agent, drilling fluid, polyester resins, insulation materials, polyester film, de-icing fluid, heat transfer fluid, automotive antifreeze, water-based adhesives, latex paints, asphalt emulsions, electrolytic capacitors, synthetic leather, polyester resin PET, jars, bottles, plastic bottles, high-strength fibers, Dacron, durable-press blends, insulated clothing, furniture filling, pillow filling, artificial silk, carpet fiber, automobile tire yarns, conveyor belts, drive belts, reinforcement for fire and garden hoses, nonwoven fabrics for stabilizing drainage ditches, culverts, railroad beds, nonwovens for diaper topsheets, disposable medical garments, high-strength plastics, magnetic recording tape, photographic film, as feedstock, solvents for cosmetics, inks, medicinal tablets, disinfectants, sterilizers, skin creams, purification of vegetable oil and fats, purification of animal oil and fats, cleaning agent, drying agent, aerosol solvent, derivative ketones, isopropylamines, isopropyl esters, propylene, polypropylene oligomers, polymerization modifier, coupling agent, heat resistant articles, kettles, food containers, disposable bottles, clear bags, flooring, mats, adhesive stickers, foam polypropylene, building materials, hydrophilic clothing, medical dressings, or any combination thereof.
